SEQUENCE LISTING (36 sequences)

SEQ ID NO: 1 - DNA, 72 bp, synthetic construct
  source 1..72: mol_type = other DNA
gttaactcggtcgccatcttgcggtgttatattgatgatgatttaaatctcatgtttgacagcttatcatcg

SEQ ID NO: 2 - DNA, 69 bp, synthetic construct
  source 1..69: mol_type = other DNA
gttaactcggtcgccatcttgcggtgttatattgatgatgatttaaatcttgaagacgaaagggcctcg

SEQ ID NO: 3 - DNA, 40 bp, synthetic construct
  source 1..40: mol_type = other DNA
ctcctccatgtccaccgtggctacatgggggtagagtcat

SEQ ID NO: 4 - DNA, 41 bp, synthetic construct
  source 1..41: mol_type = other DNA
taggtcccaacttgtatgctatgactacgtccggcgttcca

SEQ ID NO: 5 - DNA, 23 bp, synthetic construct
  source 1..23: mol_type = other DNA
agttattaatagtaatcaattac

SEQ ID NO: 6 - DNA, 17 bp, synthetic construct
  source 1..17: mol_type = other DNA
tgcagtgaaaaaaatgc

SEQ ID NO: 7 - DNA, 35 bp, synthetic construct
  source 1..35: mol_type = other DNA
gattacgccaagcttctaagcttccgggtgactca

SEQ ID NO: 8 - DNA, 31 bp, synthetic construct
  source 1..31: mol_type = other DNA
actcctcccgccccaaggccagctcaggaaa

SEQ ID NO: 9 - DNA, 33 bp, synthetic construct
  source 1..33: mol_type = other DNA
ggccttggggcgggaggagttttcttcctcgga

SEQ ID NO: 10 - DNA, 41 bp, synthetic construct
  source 1..41: mol_type = other DNA
acggccagtgaattcgatatcttgcgcaacctgctttccac

SEQ ID NO: 11 - DNA, 43 bp, synthetic construct
  source 1..43: mol_type = other DNA
aaaacgacggccagtgaattcgatatcgattcccacgccttac

SEQ ID NO: 12 - DNA, 37 bp, synthetic construct
  source 1..37: mol_type = other DNA
attgattactattaataacttcgccgtagtaccaagt

SEQ ID NO: 13 - DNA, 32 bp, synthetic construct
  source 1..32: mol_type = other DNA
gcatttttttcactgcaccaagggaaccataa

SEQ ID NO: 14 - DNA, 43 bp, synthetic construct
  source 1..43: mol_type = other DNA
gaccatgattacgccaagcttgatatccatagcccgtctgaac

SEQ ID NO: 15 - DNA, 43 bp, synthetic construct
  source 1..43: mol_type = other DNA
accatgattacgccaagcttatctgtctgcagctactttcatc

SEQ ID NO: 16 - DNA, 40 bp, synthetic construct
  source 1..40: mol_type = other DNA
atgactctacccccatgtagccacggtggacatggaggag

SEQ ID NO: 17 - DNA, 41 bp, synthetic construct
  source 1..41: mol_type = other DNA
tggaacgccggacgtagtcatagcatacaagttgggaccta

SEQ ID NO: 18 - DNA, 41 bp, synthetic construct
  source 1..41: mol_type = other DNA
ggtacccggggatcctctagagctaattgttgctctgattt

SEQ ID NO: 19 - DNA, 25 bp, synthetic construct
  source 1..25: mol_type = other DNA
gccccartgggcrtacatgcacatc

SEQ ID NO: 20 - DNA, 21 bp, synthetic construct
  source 1..21: mol_type = other DNA
agcacsccscgratgtcaaag

SEQ ID NO: 21 - DNA, 18 bp, synthetic construct
  source 1..18: mol_type = other DNA
agatggcgaccgagttaa

SEQ ID NO: 22 - DNA, 22 bp, synthetic construct
  source 1..22: mol_type = other DNA
agatgtgctgatagtagcgtag

SEQ ID NO: 23 - DNA, 40 bp, synthetic construct
  source 1..40: mol_type = other DNA
ttcacccacctttgaaactcgaattcactggccgtcgttt

SEQ ID NO: 24 - DNA, 41 bp, synthetic construct
  source 1..41: mol_type = other DNA
tagcgttagttttgccattttctagagtcgacctgcaggca

SEQ ID NO: 25 - DNA, 40 bp, synthetic construct
  source 1..40: mol_type = other DNA
agacaccgactctagatatcatgactgatgtcgagcccgc

SEQ ID NO: 26 - DNA, 40 bp, synthetic construct
  source 1..40: mol_type = other DNA
ttgtagtcagcccgggatccttatagatgaaagtagctgc

SEQ ID NO: 27 - DNA, 2502 bp, synthetic construct
  source 1..2502: mol_type = other DNA
atctgtctgcagctactttcatctataatggccgacctagaatttgacggagtgcaatctgagcaaagggctatacacttccaacgtcagtcggaccgcgaacgcaaaaacagagagctgcaaaccatacaaaacacccaccaatgtaaacgcgggatattttgtattgtaaagcaagctaagctccactacgagcttctatctggcaacgaccacgagctccaatacgtggtcgaacagcagcgtcaaacctgtgtattcttaattggggtttcccccattaaagttacacaaaccaagggtgaaaccaagggaaccataaggtgctcctgtcacctgtcagaatgtctttacactttagttaaaaccctgtgtggcttacatgattctatcccctttaattaataaacttactttaaatctgcaatcacttcttcgtccttgtttttgtcaccatccagcagcaccaccttcccctcttcccaactttcatagcagattttccgaaaagaggcgtactttcgccacactttgaaaggaacgtttacttcactttcaagctctcccacaattttcattgcagatatgaaacgcgccaaagtggaagaaggatttaaccccgtttatccctatggatattccactccaactgacgtggctcctccctttgtagcctctgatggtcttcaagaaaacccacccggggtgttgtccttaaaaatagccaaacctctaactttcaacacctccaaagccctaagcttggcaattggcccgggtttaaaaattcaggatggtaaactagtgggggagggacaagcaattcttgcaaatttgccgctgcagatcaccaacaacacaatttcacttcgctttggaaacacacttgccctaaatgacaataatgaactgcaaacaacactccaatcttcatcacccctgaaaattaccgatcagactctgtcgctcaacataggtaacagccttgtagtaaaagatgacaaactagaaaacgcgcttaaagcatccctcccactttccattgctaacaacgccatcagcctcaacgtgggcacaggactaaccataaatggaaacactttacaagctgtgcccttaaatgctctaagtcccctaagtatttccaataataacatcagcctccgctacggcagctcactaacagtgctgaacaacgaactccaaagcaacctcacatttcacccacctttgaaactcaactctaacaactcagtttccctaaacactctatctccatttagaattgaaaatggctttttaacgctctatttgggcaccaaatctggcttgctagttgaaaacagcagcttaaaagttcagacgggctatggtctgcaagtaacagacaccaatgctctaaccttaagatacctccccccattaaccattcctgactcaggatcagaacaaggtattcttaaaataaacactggacagggcttaagtgtaagccaagctggaacgcttgaaacatccctaggaggtggattaaaacatgctgagggcaaaataaccattgatactggaaacggattaacactcagtgaaaacaaacttgccgtagcggcagggagtggcttaacattccgcgacggtgccttggtagctacagggtcatcatttacacaaaccctatggactacagcagatccatctccaaactgcaccgttctcgaggaacgtgatgctaaatttactttggctcttactattagcggaagtcaagtgctagggacagtgtctatggtaggagtaaaaggcgccctttttaataacataccatcctcttccatccaagtgcagctagactttgactctaccggagccctcttgagctcctcttcgctgaaaggatactggggatatcggcagggggtctcaatagacccttatcccataattaatgccttaaactttatgccaagttctctggcctatcccccagggcaaggtaatcaagcaaaatctaacgtgtatattcccacatatttacgaggaaacctccaaagaccaatagttttaaacatcacttttaataaccaaaacacggggttttccattagattttcatgggcaaatttaacaacaggagaaggatttgcaacaccagcatgcactttttcctacattgctgaacaacaataaactatgtaacccgaactgttattccgcctccacccttccatttcactttgtaaaccacccgctccaccttttcagcagtaaacaattgcaagtcagtaggggctacaaaacttttgggagttaaaatccacacgggttcttcacaggccaagcgagtatcagttacacttataaaaccatcactaacatcagacaaagacaaacatgagtccaaagcttccggttcaagttctgattttggttcactggcggcggggataacttcttccggagggttctccatctccttcatcagctcctccatgtccaccgtgg

SEQ ID NO: 28 - DNA, 808 bp, synthetic construct
  source 1..808: mol_type = other DNA
catagcatacaagttgggacctaatctccaggtactttctgtatgaaaaccaggtgttttcccagcagtgtctgcgatgtcttctgtccccgcgacggtagcgctccttgcagtacgcgcggaacagccactcacgcaggcccaaaagacaattctcagcggaggttgacaagttaattcccctcagtctcaaggccaacagagtttcttccacagtagcataggccaaccccaaccaggaaacacaagctgacacgtcccgttcaacgggaggacaaggaagcagaggcagaggcataggcaaaagcaataaattttattccaactgatcacgtagcacttcaaacaccaggtcacgtagatggcatcgatcgtgggtttcctgatgaaacataacagcaaggtcaaaaatcaaacgattctcaagatgattaaccaccgcaggaattaggtcttcgacgcgtatatttaaaaacaccagcaatacaaaagcccggttttctcctggatctatcatagcagcacagtcatcaattagtcccaaataattttcccgcttccaatcagatataatttgcagaataattccctgtaaatccaacccggccatggcaaaaagctcggtcagggcgctctccacgtgcatgcgcaaacacaccctcatcttgtcaatccaaaaagtcttcttcctgagaaacctgcagcaaattaagaaccgccaggttaggctcgatgcctgcatcccggagcttcattctcaacatgcactgcaaatgctccagcaaatcagagcaacaattagctctag

SEQ ID NO: 29 - DNA, 885 bp, unidentified
  source 1..885: mol_type = genomic DNA
ctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcat

SEQ ID NO: 30 - DNA, 3597 bp, synthetic construct
  source 1..3597: mol_type = other DNA
caaccgcatatttcccaccctgtacgctatctttcagcaaagccgggggcagcagcaagaactgaaaataaaaaaccgcacgctgaggtcgcttacccgaagctgtctctatcacaagagcgaagagcagctgcagcgaaccctggaggacgcagaagcgctgttccagaagtactgcgcgaccaccctaaataactgaaaagcccgcgcgcgggacatcaaaccgtctgacgtcaccagcgcgcgccaaaatgagcaaagagattcccacgccttacatgtggagttaccagccgcagatgggattagccgccggcgccgcccaggattactccacaaaaatgaactggctcagcgccggacctcacatgatttcccgcgtaaacgacattcgcgcccaccgcaatcagctattgttagaacaggctgctctaaccgccacgccccgtaataacctgaaccctcccagctggccagctgccctggtgtaccaggaaacgcctccacccaccagcgtacttttgccccgtgacgcccaggcggaagtacagatgactaattcgggcgcgcaattagcgggcggatcccggtttcggtacagagttcacggcgccgcgccctatagcccaggtataaagaggctgatcattcgaggcagaggtgtccagctcaacgacgagacagtgagctcttcgcttggtctacgaccagacggagtgttccagctcgcgggctcgggccgctcttcgttcacgcctcgccaggcatacctgactctgcagagctctgcctctcagcctcgctcgggaggaattggaccccttcagtttgtggaggagtttgtaccctcggtctactttcagcctttctccggttcgcccggccagtacccggacgagttcatccccaacttcgacgcggtgagtgactctgtggacggttatgactgatgtcgagcccgcttcagtgagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggccggactcagatctcgagctcaagcttcgaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgctctgcaatcacttcttcgtccttgtttttgtcaccatccagcagcaccaccttcccctcttcccaactttcatagcagattttccgaaaagaggcgtactttcgccacactttgaaaggaacgtttacttcactttcaagctctcccacaattttcattgcagatatgaaacgcgccaaagtggaagaaggatttaaccccgtttatccctatggatattccactccaactgacgtggctcctccctttgtagcctctgatggtcttcaagaaaacccacccggggtgttgtccttaaaaatagccaaacctctaactttcaacacctccaaagccctaagcttggcaattggcccgggtttaaaaattcaggatggtaaactagtgggggagggacaagcaattcttgcaaatttgccgctgcagatcaccaacaacacaatttcacttcgctttggaaacacacttgccctaaatgacaataatgaactgcaaacaacactccaatcttcatcacccctgaaaattaccgatcagactctgtcgctcaacataggtaacagccttgtagtaaaagatgacaaactagaaaacgcgcttaaagcatccctcccactttccattgctaacaacgccatcagcctcaacgtgggcacaggactaaccataaatggaaacactttacaagctgtgcccttaaatgctctaagtcccctaagtatttccaataataacatcagcctccgctacggcagctcactaacagtgctgaacaacgaactccaaagcaacctcacatttcacccacctttgaaactcaactctaacaactcagtttccctaaacactctatctccatttagaattgaaaatggctttttaacgctctatttgggcaccaaatctggcttgctagttgaaaacagcagcttaaaagttcagacgggctatggtctgcaagtaacagacaccaatgctctaaccttaagatacctccccccattaaccattcctgactcaggata

SEQ ID NO: 31 - DNA, 698 bp, synthetic construct
  source 1..698: mol_type = other DNA
ctaagcttccgggtgactcagtgcaataaaaattttcttggcaacaggtgtatgtgtttactttacgggcgggaagggattaggggagtataaagctggaggggaaaaatccggggctgtcagatcgagtgagaagttccatggacttgtacgagagcctagagaatctaagttctttgcgacgtttgctggaggaggcttccgacagaacctcttacatttggaggtttctgtttggttcccctctgagtcgctttctgtaccgggtgaaacgagagcacctgacggaatttgatgggcttttagagcagctgcctgggctgtttgattctttgaatctcggccaccggacgctgctagaggagaggctttttccacaattggacttctcctctccaggccgtctgtgttcagcgcttgcttttgctgtacatctgttggacagatggaacgagcagacgcagctcagcccgggctacactctggacttcttgacgctatgcctatggaagttcgggatcaggagggggaggaagctgtacgagcgcctggtggagaggcatccgtctctgcgccagcagcgtctgcaagctcaagtgctgctgagacgggaggatctggaggccatttcggaggaggagagcggcatggaagaggagaatccgagagcggggctggaccctccagcggaggagtag

SEQ ID NO: 32 - DNA, 1302 bp, synthetic construct
  source 1..1302: mol_type = other DNA
atgagcggatcaagcagccagaccgcgctgagcttcgacggggccgtgtacagcccctttctgacggggcgcttgcctgcctgggccggagtgcgtcagaatgttaccggttcgaccgtggacggacgtcccgtggatccatctaacgctgcttctatgcgctacgctactatcagcacatctactctggacagcgccgctgctgctgccgccgccacctccgccgctctctccgcagccaagatcatggctattaacccaagcctttacagccctgtatccgtggacacctcagctctggagctttaccgccgagatctagctcaagtggtggagcaactcgcagccgtgagccaacagttgcagctggtgtcgacccgagtggagcaactttcccgccctccccagtaaccgcaaaaattcaataaacagaattcaataaacagcacttgaaaaagtttaaacttgtggttgactttattcctggatagctggggggagggaacggcgggaacggtaagatctggtccatcgttcccggtcgttgaggacacggtggatcttttccaagacccgatagaggtgggtctggacgttgagatacatgggcatgagcccgtctctggggtggaggtaggcccactgcagggcctcgttttcaggggtggtgttgtaaatgatccagtcgtaggccccccgctgggcgtggtgctggaagatgtccttcagcagtaagctgatggcaacgggaagacccttggtgtaggtgttgacaaagcggttgagttgagatgggtgcatgcgggggctgatgaggtgcattttggcctggatcttgaggttggctatgttgccgcccagatcacgcctgggattcatgttatgcaagaccaccagcaccgagtaaccggtgcagcgtgggaatttgtcgtgcagcttggaagggaaagcgtggaagaatttggagacccctcggtgcccgcctaggttttccatgcactcatccatgatgatggcgatgggtccccgggaggcagcctgggcaaaaacgttgcgggggtccgtgacgtcgtagttgtggtcctgggtgagttcatcataggacattttgacaaagcgcgggcagagggtcccagactggggaatgatggttccctccggtccaggggcgtagttgccctcacagatttgcatttcccaggctttgatttcagagggagggatcatgtcaacctggggggcgatgaaaaaaatggtttctggggcgggggtgatgagctgggtggaaagcaggttgcgcaa

SEQ ID NO: 33 - DNA, 2941 bp, synthetic construct
  source 1..2941: mol_type = other DNA
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 34 - DNA, 300 bp, unidentified
  source 1..300: mol_type = genomic DNA
gccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgct

SEQ ID NO: 35 - DNA, 34092 bp, unidentified
  source 1..34092: mol_type = genomic DNA
catcatcaatataacaccgcaagatggcgaccgagttaacatgcaaatgaggtgggcggagttacgcgacctttggctcgggaacgcggaagtgggcgcggcgggtttcggggaggagcgcggggcggggcgggcgtgtcgcgcggcggtgacgcgccgggaacccggaaattgagtagtttttattcattttgcaagtttttctgtacattttggcgcgaaaactgaaacgaggaagtgaaaagtgaaaaatgccgaggtagtcaccgggtggagatctgacctttgtcgtgtggagtttacccgctgacgtgtgggtttcggtctctatttttcactgtggtttttccgggtacggccaaaggtccccattttatgactccacgtcagctgatcgctagggtatttaatgcgcctcagaccgtcaagaggccactcttgagtgccggcgagaagagttttctcctccgcgctccgccaactgtgaaaaatgaggaacttcttgctatctccggggctgccagcgaccgtagccgccgagctgctggaggacattgttaccggagctctgggagacgaacctcaggtgatttctcacttttgtgaagattttagtcttcatgatctctatgacattgatccgggcgttgaggggcaagcggatgaatggctggagtctgtggacgggttttttccggacgctatgctgctagaggctgatttgcctccatctcacaattctaacactgagcccgagtcagctgctattcctgaattgtcatcaggtgaacttgacttggcttgttacgagactatgcctccggagtcggatgaggaggacagcgggatcagtaatcccacggcttatatggtctctaaggcaattgctatactaaaagaagatgatgatgatggcgatgatggatttcaactggacgctccggcagtgccggggagagactgtaagtcttgtgaataccaccgggagcgtaccggagacccgtctatgttgtgttctctgtgctatctccgtcttaacgctgcttttgtctacagtaagtgttttgtgctttttacactgtggctttgttcagttattttttctgtgtctcatagggtgttgtttattataggtcctgtttcagatgtggaggagcctgatagtactactggaaatgaggaggaaaagccctccccgcctaaactaactcagcggtgcagacctaatattctgagaccctcgccccagcgtgtgtcatcccggaaacgtgctgctgttaattgcatagaagatttattggaagagcccactgaacctttggacttgtccttaaagcgacctcgcccgcagtagggcacgctgccagttttttctctaagcttccgggtgactcagtgcaataaaaattttcttggcaacaggtgtatgtgtttactttacgggcgggaagggattaggggagtataaagctggaggggaaaaatccggggctgtcagatcgagtgagaagttccatggacttgtacgagagcctagagaatctaagttctttgcgacgtttgctggaggaggcttccgacagaacctcttacatttggaggtttctgtttggttcccctctgagtcgctttctgtaccgggtgaaacgagagcacctgacggaatttgatgggcttttagagcagctgcctgggctgtttgattctttgaatctcggccaccggacgctgctagaggagaggctttttccacaattggacttctcctctccaggccgtctgtgttcagcgcttgcttttgctgtacatctgttggacagatggaacgagcagacgcagctcagcccgggctacactctggacttcttgacgctatgcctatggaagttcgggatcaggagggggaggaagctgtacgagcgcctggtggagaggcatccgtctctgcgccagcagcgtctgcaagctcaagtgctgctgagacgggaggatctggaggccatttcggaggaggagagcggcatggaagaggagaatccgagagcggggctggaccctccagcggaggagtaggggggataccggacccttttcctgagctggccttgggggcggtggggggctcttctgtggtacgtgaggatgaagaggggtgccaacgtggacagaagcgggaccattttgagtcctcgacttttttggctgatgtaaccgtggccctgatggcgaaaaacaggttggaggtggtgtggtacccggaagtatgggaggactttgagaaaggggacttgcacctgctggaaaaatataactttgagcaggtgaaaacatactggatgaacccggatgaggactgggaggtggttttgaaccgatacggcaaggtggctctgcgcccagactgccgctaccaggttcgcgataaggtggtcctgcgacgcaacgtgtacctgttgggcaacggcgccaccgtggagatggtggaccccagaaggggcggttttgtggccaatatgcaagaaatgtgccccggggtggtgggcttgtctggggtgactttccatagtgtgaggtttagcggcagcaattttgggggcgtggttattaccgcgaacactcctgtggtcctgcataattgctacttttttggctttagcaacacctgtgtggaattgagggtgggtggcaaagtgcgcgggtgttcattttacgcttgctggaagggggtggtgagtcagggaaaggctaaagtgtctgttcacaagtgtatgctggagagatgcaccttgggcatttccagtgagggcttcctccacgccagcgacaacgtggcttctgataacggctgcgcctttcttgtaaagggaggtgggcgcatctgtcacaatatgatatgcggccctggggatgtccccccaaagccttaccagatggttacctgcacagatggcaaggtgcgcatgctcaagcctgtgcacattgtgggccaccggcgccaccgctggccagagtttgaacacaatgtgatgacccgctgtagcttgtacctgggaggcaggcgaggagttttcatgcctagacagtgtaacctggcccactgcaacgtgatcatggaacaatccgccgctacccaggtttgctttggaggaatatttgatataagcatggtggtgtataagatcttgcgctacgatgactgtcgggctcgtactcggacctgcgactgtggagcttctcacctgtgcaacctgactgtgatgggaatggtgactgaggaggtgcgactggaccattgtcagcattcatgcctgcgggaggagttttcttcctcggacgaggaggactaggtaggtgggttgggcgtggctagcgagagggtgggctataaaggggaggtgtcggctgaggctgtcttctgtttttcaggtaccatgagcggatcaagcagccagaccgcgctgagcttcgacggggccgtgtacagcccctttctgacggggcgcttgcctgcctgggccggagtgcgtcagaatgttaccggttcgaccgtggacggacgtcccgtggatccatctaacgctgcttctatgcgctacgctactatcagcacatctactctggacagcgccgctgctgctgccgccgccacctccgccgctctctccgcagccaagatcatggctattaacccaagcctttacagccctgtatccgtggacacctcagctctggagctttaccgccgagatctagctcaagtggtggagcaactcgcagccgtgagccaacagttgcagctggtgtcgacccgagtggagcaactttcccgccctccccagtaaccgcaaaaattcaataaacagaattcaataaacagcacttgaaaaagtttaaacttgtggttgactttattcctggatagctggggggagggaacggcgggaacggtaagatctggtccatcgttcccggtcgttgaggacacggtggatcttttccaagacccgatagaggtgggtctggacgttgagatacatgggcatgagcccgtctctggggtggaggtaggcccactgcagggcctcgttttcaggggtggtgttgtaaatgatccagtcgtaggccccccgctgggcgtggtgctggaagatgtccttcagcagtaagctgatggcaacgggaagacccttggtgtaggtgttgacaaagcggttgagttgagatgggtgcatgcgggggctgatgaggtgcattttggcctggatcttgaggttggctatgttgccgcccagatcacgcctgggattcatgttatgcaagaccaccagcaccgagtaaccggtgcagcgtgggaatttgtcgtgcagcttggaagggaaagcgtggaagaatttggagacccctcggtgcccgcctaggttttccatgcactcatccatgatgatggcgatgggtccccgggaggcagcctgggcaaaaacgttgcgggggtccgtgacgtcgtagttgtggtcctgggtgagttcatcataggacattttgacaaagcgcgggcagagggtcccagactggggaatgatggttccctccggtccaggggcgtagttgccctcacagatttgcatttcccaggctttgatttcagagggagggatcatgtcaacctggggggcgatgaaaaaaatggtttctggggcgggggtgatgagctgggtggaaagcaggttgcgcaacagctgtgacttgccgcagccggtgggcccgtagatgacagctatgacgggttgcagggtgtagtttagagagctacaactgccgtcatctttcaaaagcggggccacactgtttagaagttctctaacatgtaagttttcccgcactaagtcttgcaggagacgtgaccctcctagggagagaagctcaggaagcgaagcaaagtttttaagtggtttgaggccgtcggccaagggcaagttcctaagagtttgactgagcagttccagccggtcccagagctcggttacgtgctctacggcatctcgatccagcagacctcctcgtttcgggggttggggcggctctggctgtagggaatgaggcggtgggcgtccagctgggccagggtgcggtccctccatgggcgcagggttctcttcagggtggtctcggtgacggtgaatgggtgggcccctggttgggcgctggccagggtgcgcttgaggctgaggcggctggtggcgaaccgttgcttttcgtctccctgcaagtcagccaaatagcaacggaccatgagttcatagtccaggctctctgcggcatgtcctttggcgcgaagcttgcctttggagacgtgcccgcagtttgagcagagcaagcattttagcgcgtagagttttggcgccaagaaaacggattccggggaataagcatccccaccgcagttggagcaaacggtttcgcattccactagccaggtcaactgaggatcttttgggtcaaaaaccaagcgcccgccgttttttttgatgcgtttcctacctcgagtctccatgaggcggtgcccgcgttcagtcacaaagaggctgtcggtgtctccgtagacagaggtcaggacgcgctcctccaggggggttccgcggtcctcggcgtagagaaactcgcaccactctgacataaacgcccgggtccaggctaggacgaatgaggcgatgtgggaagggtaccggtcgttatcgatgaggggatcggttttttccaaggtgtgcaggcacatgtccccctcgtccgcttccaaaaaggtgattggcttgtaggtgtatgtcacgtgatcttgtccttccgcgggggtataaaagggggcgtttcccccctcctcgtcactctcttccggttcgctgtcgccaaaggccagctgttggggtacgtaaacgcgggtgaaggcgggcatgacctgggcgctgaggttgtcagtttctatatacgaggaagatttgatggcgagcgcccccgtggagatgcccttgaggtgctcggggcccatttggtcagaaaacacaatctgtcggttatcaagcttggttgcaaaagacccgtagagggcgttggagagcaacttggcgatggagcgctgggtttggtttttttcccggtcggctttttccttggccgcgatgttgagctggacatactccctggccacgcacttccagccggggaaaacggccgtgcgctcgtccggcaccagcctcacgctccatccacggttgtgcagggtgatgacgtcgatgctggtggccacctctccccgcaggggctcgttggtccagcagaggcggccgcccttgcgagagcagaagggtggcagggggtcaagcaggcgctcgtccggggggtcggcgtcgatggtaaagatggcgggtagcaggtgtttgtcaaagtaatcgatctgatgcccgggccgacgcaggacgatttcccagtcccgcaccgccaaggcgcgctcgtagggacttagtggggcgccccagggcatgggatgcgttagggccgaggcatacatgccgcagatgtcgtaaacgtaaaggggttcctccaggatgccgaggtaggtggggtagcagcgccccccgcggatgctggcccgaacgtagtcgtagagctcatgcgagggggccagaaggtggcgactgaggtgtgcgcgctgcggtttttcatctcgaaagaggatctgcctgaagatggcgtgggagttggaagagatggtggggcgctgaaaaatgttgaagcgggcgtctggcagacccacggcctcgccaatgaagtgggcgtaggactcttgcagtttttccaccagggaggcggtgaccagcacgtccagagcgcagtagtccagggtttcccgcacgatgtcgtaacgctcttcctttttttccttccagaggtctcggttgaagagatactcttcgcggtctttccagtactcttggagaggaaacccgtcttcgtctccacggtaagagcccaacatgtaaaactggttgacggcctgatagggacagcagcccttctccacgggcagcgagtaggccagggcggccttgcgcagggaggtgtgagtcagggcaaaggtgtcgcggaccatgacttttacaaactggtacttgaagtcccggtcgtcgcacatgcctcgctcccagtctgagtagtctgtgcgctttttgtgtttggggttaggcagggagtaggtgacgtcgttaaagaggattttgccgcatctcggcataaagttgcgagagattctgaagggtcccggcacctccgagcggttgttgatgacttgggcggccaggagaatctcgtcgaagccgttgatgttgtgccccacgacgtaaaactcgatgaaacgcggtgcgctgcgcagcagggggcacttttccagttgctggaaagtaagttcccgcggctccacgccgtgttccgtgcggctccagtcctccaccgggtttcgctccacaaaatcctgccagatatggtcgaccagcgagagctgcaggcggtcgcgaaactcgcggaatttcctgccgatggcttgcttctgggggttcaagcaaaaaaaggtgtctgcgtggtcgcgccaggcgtcccagccgagctcgcgagccagattcagggccagctgcaccagagccggctcgccggtgattttcatgaccaggagaaagggcaccagctgttttccgaacgcgcccatccaggtgtaggtctccacgtcgtaggtgagaaacagacgttcggtccgcgggtgcgatcccagggggaaaaacttgatgggttgccaccattgggagctctgggcgtggatgtgatggaagtaaaagtcccggcggcgcgtggaacattcgtgctggtttttgtaaaagcggccgcagtggtcgcagcgcgagacggagtgaaggctgtgaatcaggtgaattttgcgccgctgagggggccccagagccaaaaatcggagcggaaacgaccgcgcggccacctcggcgtccgcaggcaagatggatgagggttccaccgttccccgtcctcggaccgaccacacttccgccagctgcggcttcagttcttgtaccagcgcccgaagcgtttcgtcgctgggcgaatcgtgaatacggaagtggtcgggcagaggcgggaggcggtggacttccaggaggtgtgtgagggccggcaggagatgcaggtggtacttgatttcccacggatggcgatcgcgggcgtccaaagcgaagagatggccgtggggccgcggcgccaccaacgttccacggggtgtttttatcggcggcggggacgggctcccggaggcagcggcggctcgggtcctgccggcaagtcgggcagcggcacgtcggcgtggagctcgggaagaggctggtgctgcgcgcggagctgactggcaaaggctatcacccggcgattgacgtcctggatccggcggcgctgcgtgaataccaccggacccgtggtcttgaacctgaaagagagttcgacagaatcaatctcggcatcgttaaccgcggcctggcgcagtatttcggccacgtccccggagttgtcttgatacgcgatttctgccatgaactggtcgatttcctcttcctgcaagtctccgtgaccggcgcgttcgacggtggccgcgagatcgttggagatgcggcccattagctgggaaaaggcattgatgccgacctcgttccacactcggctgtacaccacctctccgtgaacgtcgcgggcgcgcatgaccacctgggcgagattgagttccacgtggcgggcgaaaaccggatagtttcggaggcgctgatacagatagttgagggtggtggcggcgtgctcggccacgaaaaaatacatgatccagcggcggagggtcagctcgttgatgtccccgagcgcctccaggcgttccatggcctcgtaaaagtccacggcaaagctgaaaaattggctgttcctggccgaaaccgtgagctcttcttccaagaggcgaatgagatccgccacggtggccctgacttcgcgttcgaaagccccgggtgcctcctcctcctcgacttcttcgaccgcttcgggcacttcctcttcctcgaccaccacctcaggcggggctcggcggcgccggcggcggacgggcaggcggtcgacgaaacgctcgatcatttcccccctccgtcgacgcatggtttcggtgacggcgcgaccctgttcgcgaggacgcagggtgaaggcgccgccgccgagcggaggtaacagggagattggggggcggtcgtgggggagactgacggcgctaactatgcatctgatcaatgtttgcgtagtgacctcgggtcggagcgagctcagcgcttgaaaatccacgggatcggaaaaccgttccaggaacgcttctagccaatcacagtcgcaaggtaagctgaggaccgtctctggggctggtctgttctgtcttccagcggtggtgctgctgatgaggtagttgaagtaggcgctcttgaggcggcggatggtggacaggagaaccacgtctttccgtccagcttgctgtatccgcaggcggtcggccataccccacacttctccttgacagcggcggaggtccttgtagtattcttgcatcagcctttccacgggcacctcgtcttcttcttccgctcggccggacgagagccgcgtcaagccgtacccgcgctgcccctgtggctggagcagggccaggtcggccacgacgcgctcggccagcacggcctgctggattctggtgagtgtgtcctggaagtcgtcgaggtccacgaagcggtgatacgcgccggtgttgatggtgtaggtgcagttgctcatgacggaccagttcacggtctgggtgccgtggcccacggtttccaggtagcggagacgcgagtaggcccgtgtctcgaagatgtagtcgttgcaggtccgcagcaggtactggtagcccaccagcagatgcggtggcggctggcggtaaaggggccaccgctgggtggcgggggcgttgggggcgagatcttccaacatcaggcggtgatagccgtagatgtagcgcgacatccaagtgatgccgctggccgtggtgctggcgcgggcgtagtcgcgaacgcggttccagatgttgcgcagcggctggaagtactcgatggtgggtcgactctgccccgtgaggcgggcgcagtcggcgatgctctatggggaaaaagaagggccagtgaacaaccgccttccgtagccggaggagaaacgcaaggggcaaagaccaccgaggctcgggttcgaaacccggtcggcggccccgaatacggagggcggtttttgcttttttctcagatgcatcccgtgctgcggcagatgcgtccgaacgcggggtgccagtccccggcggtgtctgcggccgtgacggcggcttctacggccacgtcgcgctccaccccacctactacggcccaggcggcggtggctctgcgcggcgcgggggaacccgaagcagaggcggtgttggacgtggaggagggccaggggttggcgcggctaggggccttgagtcccgagcggcacccgcgcgtggctctgaagcgcgacgcggcggaggcgtacgtgccgcggagcaatctgtttcgcgaccgcagcggcgaggaggccgaggagatgcgagacttgcgttttcgggcggggcgggagctgcggcacgggttggaccggcagagggttctgagagaggaggactttgaggcggacgagcgcacgggggtgagtcccgcgcgggctcacgtggcggccgccaacctggtgagcgcgtacgagcagacggtcaaggaggagatgaacttccagaagagtttcaatcatcacgtgcgcaccctaatcgcgcgcgaagaggtggctataggcctcatgcatctgtgggattttgtggaggcgtacgttcagaatcccagcagcaagccgctgacggctcagctcttcctcatcgtgcaacacagccgggacaacgagacgtttagggaggccatgctgaacatcgcggagccggagggacgctggctcttggatctcattaacatcttgcagagtatcgtagtgcaggagcgctcgctgagcctggccgacaaagtggccgccatcaactacagcatgttgtcgctgggtaaattttacgcccgcaagatttacaagtctccattcgttcccattgacaaggaggtgaagatagacagcttttacatgcgcatggcgctcaaggtgctgactttaagcgacgacttgggggtgtaccgcaacgaccgcatccacaaggcggtgagcgccagccgccggcgcgagctgagcgaccgcgagcttttgcacagcctgcatcgggcgttgactggcgccggcagcgccgaggcggccgagtactttgacgccggagcggacttgcgctggcagccatcccgacgcgcgctggcggcggccggcgtcggggagtacggggtcgaggacgacgatgaagcggacgacgagtcgggcattgacatgtagccgtttttcgttagatatgtcggcgaacgagccgtctgcggccgccatggtgacggcggcgggcgcgccccaggacccggccacgcgcgcggcgctgcagagtcagccttccggagtgacgcccgcggacgactggtccgaggccatgcgtcgcatcctggcgctgacggcgcgcaaccccgaggcttttcggcagcagccgcaggcgaaccggtttgcggccattttggaagcggtggtgccctccagacccaaccccacccacgaaaaggtgctggccatcgtcaacgccctggcggagaccaaggccatccgcccagacgaggccgggcaggtttacaacgcgctgctagaaagggtggggcgctacaacagctccaacgtgcagaccaatctggaccgcttggtgacggacgtgaaggaggccgtggcccagcgagagcggtttttcaaggaggccaatctgggctcgctggtggccctcaacgccttcctgagcacgctgccggcgaacgtgccccgcggccaggaggattacgtgaactttctgagcgccctccgcctgatggtggccgaggtgccgcagagcgaggtgtaccagtctggccccaactactacttccagacctctcgccagggcctgcagacggtgaacctgacgcaggcctttcagaatctgcagggcctctggggggtgcgcgctccgctgggcgaccgcagcacggtgtccagcctgctgacccccaacgcccgcctgctcttgcttctcatcgctccgttcaccgacggcggttccatcagccgcgactcttacctgggacacctgctcaccctctaccgcgaggctattggccaggcgcgggtggacgagcagacgtaccaggagatcaccagcgtgagccgcgcgctggggcaggaggacactggcagcttggaggcgactttgaacttcctgctgaccaaccggcgacagcgcctaccgccccagtacgcgctgaactctgaggaggagcgcatcctgcgtttcgtgcagcagagtaccgcgctgtacttgatgcgtgaaggcgcttctcccagcgcttcgctggatatgacggcggcaaacatggagccttctttctacgccgccaaccgccccttcgttaaccgactgatggattatttgcatcgggcagcggccctgaaccctgaatactttactaacgtcatcctgaacgaccgttggctgccacctcccggcttttacacgggggagttcgacctcccggaggccaacgacggtttcatgtgggacgacgtggacagcgtgtttttagccggcaagaaagaggcgggagactctcagagccagcgcgcgagcctctcggacttgggggcggcagggccggcgtctccgctgccccgcctgccaagcgccagcagcgccagcgtggggcgggtgagccgtccgcgccttagcggcgaggatgactggtggaacgacccgctgctgcgcccgtcccgcaacaaaaacttccccaacaacgggatagaggatttggtagacaaaatgaaccgttggaagacgtatgcccaggagcatcgtgagtggcaggcgcggcaaccgatgggccctgtgctgcctccctcttggcgaccgcgcagggacgaagacgccgacgattcagccgatgacagcagcgtgttggatctgggcgggagcgggaacccctttgcccacctgcagcctcgcggcgtgggtcggcggtggcgctaggaaaaagatcattaaagcacttaccagagccatggtaagaagagcaacaaaggtgtgtcctgctttcttcccggtagcaaaatgcgtcgggcggtggcagttccctccgcggcaatggcgttaggtccgcccccttcttacgaaagcgtgatggcggcggccaccctgcaagcgccgttggagaatccttacgtgccgccgcgatacctggagcccactggcgggagaaacagcattcgttactcggagctgacgcccctgtacgacaccacccgcctgtacctggtggacaacaagtcagcagatatcgccaccttgaactaccagaacgaccacagcaactttctcacgtccgtggtgcagaacagcgactacacgcccgccgaagcgagcacgcagaccattaacttggacgaccgctcgcgctggggcggggacctgaaaaccattctgcacactaacatgcccaacgtgaacgagttcatgtttacaaactcgttcagggctaaacttatggtggcgcacgaggccgacaaggacccggtttacgagtgggtgcagctgacgctgccggaggggaacttttcagagattatgaccatagacctgatgaacaacgccatcattgaccactacctggcggtggccagacagcagggggtgaaagaaagcgagatcggcgttaagtttgacacgcgcaattttcgactgggctgggacccggagacggggctggtgatgccgggggtgtacacgaacgaagccttccatcccgacgtagtactcttgccaggctgcggggtggacttcacttacagtcggttaaacaacctgctgggcatacgcaagagaatgccctttcaggaagggtttcagatcctttacgaggacctggagggcggtaacattccggccctgctggacgtgccggcgtacgaggagagcatcgcgaacgcaggggagacggcgatcaggggcgataatttcgcggcgcagccacaggcggctccaaccataaaacccgttttggaagactctaaagggcggagctacaacgtaatagcagacaccaataacacggcgtacagaagctggtatctggcttacaactacggcgaccatttgaagggggtgagggcctggaccctgctcaccactccggacgtgacgtgcggttccgagcaggtttactggtcgctgccggacatgtacgtcgacccagtgacattccgctccacgcaacaagtgagcaactacccagtggtgggagcggagctgatgcccattcacagcaagagtttttacaacgagcaggccgtttactcgcagctcatccgtcagaccaccgccctaacgcacgttttcaaccgcttccccgagaaccaaatcctagtgcgacccccagcgcccaccatcaccaccgtcagcgagaacgtgcccgctctaaccgatcacgggacgctgcctttgcagaacagcatccgcggagttcagcgagttaccatcacggacgcccgtcgtcggacctgtccctacgtctacaaagccttgggaatcgtggccccgcgcgtcctgtcgagtcgcactttctagatgtccatcctcatctctcccagcaacaataccggttggggcctgggcgtgaccaaaatgtacggaggcgccaaacgacggtccccacagcatcccgtgcgagtgcgcgggcattttagagccccatgggggtcgcacacacgcgggcgcaccggccgaaccaccgtcgacgacgtgatcgatagcgtggtggccgacgcccgcaactaccaacccgctcgatctacggtcgacgaagtcatcgacggcgtggtggcagacgccagggcctacgcccggagaaagtctcgtctgcgccgccgccgttcgttaaagcgccccacggccgccatgaaagccgctcgctctctcctgcgtcgcgcgcgtatcgtgggccgtcgcgccgccagacgcgcagccgccaacgccgccgccggccgagtgcgccgccgggccgcccagcaggccgccgccgccatctccagtctggccgccccccgtcgcgggaacgtgtactgggtgagggactcggccaccggcgtgcgagttccagtcagaacccgtcctcctcgttactgaataaaaaaagttctaagcccaatcggtgttccgttgtgtgttcagctcgtcatgaccaaacgcaagtttaaagaggagctgctgcaagccctggtccccgaaatctatgcgccggcgccggacgtgaaaccgcgtcgcctgaaacgcgtgaagaagcaggaaaagctagagacaaaagaggaggaagcggtggcgttgggagacggggaggtggagtttgtgcgctcgttcgcgccgcgtcggcgggtgaattggaaagggcgcaaggtgcagcgggtgctgcgccccggcacggtggtgtctttcactcccggcgaaaagtccgcctggaagggaattaagcgcgtgtacgatgaggtgtacggggacgaggacattctcgagcaggcgctggacagaagcggggagtttgcttacggcaagagggcgaggacgggcgacatcgccatcccgctggacacttccaaccccacccccagtctgaaacccgtgacgctgcagcaggtgttgccggttagcgccccctcgcgacgcggcatgaaacgcgagggcggcgagctccagcccaccatgcagcttctggttcccaagaggcagaaactagaggacgtgctggacaggataaaaatggagcccgacgtgcagcccgacattaaaatccgacccatcaaagaagtggcaccgggaatgggcgtgcagaccgtggacatccagattcccatgaccagcgcggctcaggcggtagaggccatgcagaccgacgtggggatgaagacagacctgcccgcagccgccgctgccgccgtggccagcgccgcgacgcagacggatgccggcatgcagaccgacccgtggacggaggcgcccgtgcagccggcgagaagacgcgccagacggacgtacggacctgtttctggcgtgatgccggagtacgcgttgcatccttccatcatccccacccccggctatcggggacgcacttaccgcccgcgacgcagcaccacgcgccgccgtcgccgtaccacgcgggtcgtccccgccagagtgagacgcgtaaccacacgccgcggacgccgtttgactctgcccgtggtgcgctaccatcccagcattctttaaaaaaccgctcctacgttgcagatgggcaagcttacttgtcgactccgtatggccgtgcccggctaccgaggaagatcccgccgacgacggactttgggaggcagcggtttacgccgccgtcgggcggttcaccggcgcctcaagggaggcattctgccggccctgatccccataatcgccgcagccatcggggccattcccggaatcgccagcgtagcggtgcaggctagccagcgccactgattttactaaccctgtcggtcgcgccgtctctttcggcagactcaacgcccagcatggaagacatcaatttctcctctctggccccgcggcacggcacgcgcccgtatatggggacgtggagcgagatcggcacgaaccagatgaacgggggcgctttcaattggagcagtgtgtggagcggcttgaaaaatttcggctccactctgaaaacttacggcaaccgggtgtggaactccagcacggggcagatgctgagggacaagctaaaggactcgcagtttcagcaaaaggtggtggacggcattgcttcgggcctcaacggcgccgtcgacctggccaatcaggccattcaaaaggaaattaacagccgtctggagccgcggccgcaggtggaggagaacctgccccctctggaggcgctgccccccaagggagagaagcgcccgcggcccgacatggaggagacgttagttactaagagcgaggagccgccttcgtacgaggaggcggtgggtagctcgcagctgccgtccctcacgctaaagcccactacctatcccatgaccaagcccatcgcctccatggcgcgccccgtgggagtcgacccgcccattgacgcggtggccactttggacctgcctcgtcccgaagccagcagccgggtgcccccagttcccatcgctccgccggtttctcgccccgccatccgccccgtcgccgtggccactccccgctatccgagccgcaacgccaactggcagagcaccctgaacagtattgtcggactgggagtgaagtctctgaagcgccgtcgctgtttttaaaaatttttattaaacgagtagccctgtcttaatccatcgttgtatgtgtgcctatatcacgcgttcagagcctgaccgtccgtcaagatggccactccgtcgatgatgccgcagtggtcgtacatgcacatcgccgggcaggacgcctcggagtacctgagcccgggtctggtgcagtttgcccgtgcgacggaaacctacttctcactgggcaacaagttcaggaaccccaccgtggcgcccacccacgacgtcaccaccgatcggtcccagcgactgacaatccgcttcgtccccgtggacaaggaagacaccgcttactcctacaaaacccgcttcacgctggccgtgggcgacaaccgggtgctagacatggccagtacctactttgacatacgcggcgtgatcgaccgcggacctagcttcaagccgtactcgggcacggcttacaactcactggctcccaaaggggcgcctaacaacagccaatggaacgccacagataacgggaacaagccagtgtgttttgctcaggcagcttttataggtcaaagcattacaaaagacggagtgcagatacagaactcagaaaatcaacaggtcgctgccgacaaaacttaccagccagagcctcaaattggagtttccacctgggataccaacgttaccagtaacgctgccggacgagtgttaaaagccaccactcccatgctgccatgttacggttcatatgccaatcccactaacccaaacggggggcaggctaaaacagaaggagaaatttcgctaaactttttcacaacaactgcggcagcagacaataatcctaaagtggttctttacagcgaagatgtaaaccttcaagcccccgatactcacttagtatataagccaacggtaggagaaaacgttatcgctgcagaagccctgctaacgcagcaggcgtgtcccaacagagcgaactacataggcttcagagataactttatcggtttaatgtattataacagcacagggaacatgggagttctggcaggtcaggcctcgcagttaaacgccgttgtagacctgcaagatcgaaacacggaactgtcttatcagctgatgctagatgctctgggtgacagaactcgatatttctcaatgtggaatcaggccgtggacagctacgatccagacgttaggattatcgagaaccatggggtggaagacgagctgcccaattactgtttcccactcccgggcatgggcatctttaattcctacaagggggtaaaacctcagaatggcggtaatggtaactgggaagcaaacggggacctgtcaaatgccaatgagattgctttaggtaacattttcgccatggagattaacctccacgcaaacctgtggcgcagctttttgtacagcaatgtggcgctgtacctgccagacagctataaattcactcccgctaacataactctgcccgccaaccaaaacacctacgagtacattaatgggcgcgtcacttctccgaccctggtggacacctttgttaacattggagcccgatggtcgccggatcccatggacaacgtcaacccctttaaccatcaccggaacgccggcctccgataccgctctatgctgctgggaaatggccgcgtggtgcctttccacatacaagtgccgcagaaatttttcgcaattaaaaacctcctgcttttgcccggctcctacacttacgagtggagcttcagaaaagacgtgaacatgatcctgcagagcaccctgggcaatgaccttcgagtggacggggccagcgttcgcattgacagcgtcaacttgtacgccaactttttccccatggcccacaacacagcttctaccttggaagccatgctgcgaaacgacaccaacgaccagtcgtttaacgattacctcagcgcggccaacatgctttatcccattccggccaacgccacaaacgttcccatttctattccctcccgcaactgggcggctttccgcgggtggagcttcacccgccttaaagccaaagaaacgccttccttgggctccggctttgacccctactttgtgtactcgggcaccattccttatctggacggcagcttttacctcaaccacaccttcaaacgtttgtccatcatgttcgattcttccgtaagttggccgggcaatgatcgccttcttacgccgaacgagttcgaaattaagcgcattgtggacggggaaggctacaacgtggctcaaagtaacatgaccaaagactggtttttaattcagatgctcagccactacaacatcggctaccaaggcttctatgttcccgagggctacaaggatcggatgtattctttcttccgaaactttcagcccatgagccgccaggtgccggatcccaccgctgccggctatcaggccgttcccctgcccagacaacacaacaactcgggcttcgtggggtacatgggcccaaccatgcgcgaaggacagccctacccggccaactatccctatccgctgattggagctaccgcagtccccgccattacccagaaaaagttcttgtgcgaccgcgtcatgtggcgcatacctttttccagcaactttatgtcgatgggggccctgaccgacctcggtcagaatatgctttacgccaactccgcccatgccctggatatgacttttgaggtggaccccatgaacgagcccacgttgctgtatatgctttttgaagtgtttgacgtggtcagagtgcaccagccgcaccgcggtatcatcgaggccgtgtacctgcgcacccccttctccgcgggcaatgccaccacctaagccgctgaactagctggtttttaccccagatcccatgggctccacggaagacgaactgcgggccattgtgcgagacctgggctgcggaccctacttcctgggcacctttgacaagcggtttcccgggttcgtgtctcctcgcaaactcgcgtgcgcgatcgtgaataccgccggccgagagaccggaggagagcattggctagctctgggctggaacccccgctcctccacgtttttcctgtttgacccctttggcttttcagaccaacgcttgaagcagatctatgcctttgaatatgagggactgctcaagcgaagcgcgctggcctcctccgccgatcactgtttaaccctggtaaagagctctcagacggttcagggccctcacagcgccgcctgtggtcttttttgttgcatgtttttgcatgcctttgtgaactggccggacacccccatggaaaacaaccccaccatggacctcctgactggcgtgcccaactccatgctccaaagccccagtgtgcaaaccaccctcaaccaaaaccagaaaaacctatacgccttcctgcaaaaacattctccctactttcgccgccatcgggaacagatagaaaatgcaaccgcgtttgacaagactctgtaacgtttaataaatgaacttttattgaactggaaaactgggtttgtgattttttaaaaatcaaaggggtttagctggacatccatgtgggaggccggcagggtggtgttcttgtactggtacttgggcagccacttgaactcgggaatcacaaacttgggcagcggtatttctgggaagttgtcgtgccacagctggcgggtcagctgcagtgcttgcagaacatcgggggcggaaatcttgaagtcgcagtttatctggttcacggcacgcgcgttgcggtacatgggattggcacactgaaacaccagcaggctgggattcttgatgctagccagggccacggcgtcggtcacgtcgccggtgtcttctatgttggacagcgaaaaaggcgtgactttgcaaagctggcgtcccgcgcgaggcacgcaatctcccaggtagttacactcacagcggatgggcagaagaagatgcttgtggccgcgggtcatgtagggataggctgcagccataaaagcttcgatctgtctgaaagcttgcttggccttgtggccttcggtgtaaaaaacaccgcaggacttgttggaaaaggtattactggcgcaagcggcatcgtgaaagcaagcgcgagcgtcttcgtttcgtaactgcaccacgctgcggccccaccggttctgaattaccttcgccctgccggggttctccttgagagcgcgctggccggcttcgctgcccacgtccatttccacgacgtgctccttgttaatcatggccagaccgtggaggcagcgcagctcctcgtcgtcgtcggtgcagtgatgctcccacacgacgcagccagtgggctcccacttgggcttggaggcctcagcaatgccagaatacaggagaacgtagtggtgcagaaaacgtcccatcatggtgccaaaggttttctggctgctgaaggtcagcgggcagtacttccagtcctcgttaagccaagtgttgcagatcctcctgaagaccgtgtactgatcgggcataaagtggaactcattgcgttcggtcttgtcgatcttatacttttccatcagactatgcataatctccatgcccttctcccaggcgcaaacaatcttggtgctgcacgggttagggatggccaaagtagtgggcctctgaggcggcgcttgttcttcctcttgagccctctcccgactaacgggggttgaaagagggtgccccttggggaacggcttgaacacggtctggcccgaggcgtcccgaagaatctgcatcgggggattgctggccgtcatggcgatgatctgaccccggggctcctccacttcgtcctcctcgggactttcctcgtgcttttcgggggacggtacgggagtagggggaagagcgcggcgcgccttcttcttgggcggcagttccggagcctgctcttgacgactggccattgtcttctcctaggcaagaaaaacaagatggaagactctttctcctcctcgtcaacgtcagaaagcgagtcttccaccttaagcaccgagaactcccagcgcgtagaatccgatgtgggctacgaaactccccccgaaaacttttcgccgccccccataaacactaacgggtggacggactacctggccataggagacgtactgctgaaacacatcaggcgacagagcgtgatcgtgcaagatgctctcaccgagcgactcgcggttccgctggaagtggcggaacttagcgcggcctacgagcgaaccctattctccccaaagactccccccaagaggcaggctaacggcacctgcgagcctaaccctcgactcaacttctaccctgcctttgccgtgccagaggtgctggctacgtaccacatttttttccaaaaccacaaaatccctctctcgtgccgcgccaaccgcaccaaagccgatcgcgtgctgcagctaggggaaggggctcgcatacctgagattgcgtgtctggaagaagtcccaaaaatctttgaaggtctgggtcgagacgaaaagcgagcagcaaacgctctggaagagaacgcagagagtcacaacagtgccttggtagaactcgagggcgacaacgcccgactggccgtccttaaacggtccatagaagtcacacactttgcctaccccgccgttaacctccctccaaaagttatgacagcggtaatggactcgctgcttataaagcgcgctcagcccttagacccggagcacgaaaacgacagtgacgaaggaaaaccggtggtttctgatgaggagttgagcaagtggctgtcctctaacgaccctgtcacgttggaggaacgaagaaaaaccatgatggccgtggtgctggttaccgtgcaattagaatgtctgcagaggttcttttcccacccagaaaccctgagaaaagtggaggaaacgctgcactacacatttaggcacggctacgtgaagcaagcctgcaagatttccaacgtagaacttagcaacctcatctcctacctggggatcttgcacgaaaaccgcctcggacagaacgtgctgcacagcacactgaaaggagaagcccgccgagactatgtgcgagactgcgtgttcctagcgctagtctacacctggcagagcggaatgggagtatggcagcagtgcctggaggacgaaaacctcaaagagcttgaaaagctgctggtgcgctccagaagagcgctgtggaccagttttgacgagcgcaccgccgcgcgagacctagctgatattattttccctcccaagcttgtgcaaacgcttcgggagggtctgcccgatttcatgagccaaagcatcttgcaaaacttccgctcttttatcttggaacgctcgggaatcttgcccgccactagctgcgccctacccacagattttgtgcctcttcactaccgcgaatgtccaccgccgctgtggccgtacacttacttgcttaaactggccaactttttaatgttccactctgacctggcagaagacgtgagcggcgaagggctgctagaatgccactgccgctgcaacctgtgcaccccccaccgatccctagtatgcaacactccccttctcaatgagacccagatcatcggtacctttgaaatccagggaccctccgacgcggaaaacggcaagcaggggtctgggctgaaactcacagccggactgtggacctccgcctatttgcgcaaatttgtaccagaagactatcacgcccaccaaattaaattttacgaaaaccaatcgaaaccacccaaaagcgagttaacggcttgcgtcatcacgcagagcagcatagttggacagttgcaagccattaacaaagcgcggcaagagtttctcctaaaaaaaggaaaaggggtctacttggacccccagaccggcgaggaactcaacggaccctcctcagtcgcaggttgtgtgccccatgccgcccaaaaagaacacctcgcagtggaacatggcagagacggaggaagaggagtggagcagtgtgagcaacagcgaaacggaggaagagccgtggcccgaggggagcaacggggaagaggacacggaggacggcgaagtcttcgccgaagaactctcgccactacccccgaagtcccagccagccgcctcggcccaagagcccgcacacacccgtagatgggatagcaagaccaaaaagccgggtaagagaaacgttcgcccccgccagggctaccgctcgtggagaaagcacaaaaactgcatcttatcgtgcctcctccagtgcggcggagacgtttcgttcacccgtagatacttgctttttaacaaaggagtggccgttccccgtaacgtcctccactactaccgtcactcttacagctccgaagcgaacggctaagaaaacgcaactgccggctggaggactgcgtctcagcgccagagaaccgccagccaccagggagctccgaaaccgcatatttcccaccctgtacgctatctttcagcaaagccgggggcagcagcaagaactgaaaataaaaaaccgcacgctgaggtcgcttacccgaagctgtctctatcacaagagcgaagagcagctgcagcgaaccctggaggacgcagaagcgctgttccagaagtactgcgcgaccaccctaaataactgaaaagcccgcgcgcgggacatcaaaccgtctgacgtcaccagcgcgcgccaaaatgagcaaagagattcccacgccttacatgtggagttaccagccgcagatgggattagccgccggcgccgcccaggattactccacaaaaatgaactggctcagcgccggacctcacatgatttcccgcgtaaacgacattcgcgcccaccgcaatcagctattgttagaacaggctgctctaaccgccacgccccgtaataacctgaaccctcccagctggccagctgccctggtgtaccaggaaacgcctccacccaccagcgtacttttgccccgtgacgcccaggcggaagtacagatgactaattcgggcgcgcaattagcgggcggatcccggtttcggtacagagttcacggcgccgcgccctatagcccaggtataaagaggctgatcattcgaggcagaggtgtccagctcaacgacgagacagtgagctcttcgcttggtctacgaccagacggagtgttccagctcgcgggctcgggccgctcttcgttcacgcctcgccaggcatacctgactctgcagagctctgcctctcagcctcgctcgggaggaattggaccccttcagtttgtggaggagtttgtaccctcggtctactttcagcctttctccggttcgcccggccagtacccggacgagttcatccccaacttcgacgcggtgagtgactctgtggacggttatgactgatgtcgagcccgcttcagtgctagtggaacaagcgcggctcaagcacctggtccgttgccgccgccgccgctgcgtggctcgcgacttgagcttagctctcaagtttgttaaaaacccgtccgaaaccgggagcgctgtgcacgggttggagctagtgggccctgaaaaggccacaatccacgttctcagaaactttgtggagaaacccattttggttaaacgagatcaggggccttttgcaatcagcctactctgcacctgtaaccatgttgatcttcacgactattttatggatcatttgtgtgctgaattcaataagtaaagcaaaatctaaccaagattataatggccatgactgttcctatccaagatatgacgtagcgccggtaaactcactttccaacttatctctgaactgttcattttggtccgcacagcttacttggtactacggcgacagcgttctttccggctccttgggcaaatcacacggaataactcttcagctcttttctccgtttcgatacggaaactacagctgtcaggccggaccctgccttcacgtctttaatctccagccttgtccaccaactaaacttgtcttcgccgaatccaagcacctacagcttaactgcagcctcacaggcccacgtatcttgtggacatacaataaatacaggttggtggaatttgtctactacccgcccagcgcccacggttttggggaaattccttcccagctctactacccctattttgacactcattttgcaagccaacaacaactgcacttgcagtctcccttcgccccaggagagtactcttgccacgtaggctcctgcaaagaaacttttattctcttcaacagatcttccaacattgaacgcttcactactaactactttagaaaccaagttgtgcttttcactgacgaaacccctaacgtcaccctggactgtgcatgtttttcccatgacaccgtaacttggactcttaacaatactctctggctcgcgttcgataaccaaagcttgtttgttaaaaattttgatttaacctttactaaaccctctcctcgcgaaatagttatctttgcgcctttcaatccaaaaactaccttagcctgtcaggttttgtttaaaccctgccaaacaaactttaagtttgtttatttgcctccgcaatctgtcaaactcatagagaaatacaacaaagcgcccgtcttagcccctaaaaccttctaccactggttaacatacgcggggctatttgcgctaattgtttttttcctaattaacatttttatatgtttttcaccttcctccttcttttcgcgaacaccgttgccgcagaaagacctctccttattactgtagcgcttgctatacaaaaccaagagtggttaaccgttctctcaatctattttcaatttttcattttgtccctaatactttctctcattgttgttaacaatgatctggagcattggtctcgcctttttttggctgcttagtgcaaaagccaccatttttcacaggtatgtggaagaaggaactaacaccctctttaccatacctgaaacaattaaagcggctgataaagtttcttggtacaaaggctcgctctcagacggcaaccactcattctcaggacagactctctgcattcaagaaacttattttaaatcagaactacaatacagctgcataaaaaattttttccatctctacaacatctcaaaaccctatgagggtatttacaatgccaaggtttcagacaactccagcacacggaacttctactttaacctgaccgtcattaaagcaatttccattcctatctgtgagtttagctcccagtttcttaccgaaacctactgtttaattactataaactgcactaaaaatccccttcacaccaccataatctacaatcacacacaatcaccttgggttttaaacctaaaattctctccacgcatgccttcacaattcctcacgcaagttaccgtctctaacataagcaagcagtttggctttcactatcctttccacgaactgtgcgaaataattgaagctgaatatgaacccgactactttacttacattgccctcggtgtaatcgttgtttgcctttgctttgttattgggggatgtgtttatttgtacattcagagaaaaatattgctctcgctgtgctcctgcggttacaaagcagaagaaagaattaaaatctctacactttattaatgttttccagaaatggcaaaactaacgctactccttttgcttctcacgccggtgacgctttttaccatcactttttctgccgccgccacactcgaacctcaatgcttgccaccggttgaagtctactttgtctacgtgttgctgtgctgcgttagcgtttgcagtataacatgttttacctttgtttttcttcagtgcattgactacttctgggtcagactctactaccgcagacacgcgcctcagtatcaaaatcaacaaattgccagactactcggtctgccatgattgtcttgtattttaccctgattttttttcaccttacttgcgcttgtgattttcacttcactcaattttggaaaacgcaatgcttcgacccccgcctctccaacgactggatgatggctgttgcaatagccacgcttggggcgtttggactttttagtggttttgctttgcattacaaatttaagactccatggacacatggctttctttcagattttccagtcacacctactccgccacctcccccggccatcgacctgcctcaagttccctcaccttctccatctgtctgcagctactttcatctataatggccgacctagaatttgacggagtgcaatctgagcaaagggctatacacttccaacgtcagtcggaccgcgaacgcaaaaacagagagctgcaaaccatacaaaacacccaccaatgtaaacgcgggatattttgtattgtaaagcaagctaagctccactacgagcttctatctggcaacgaccacgagctccaatacgtggtcgaacagcagcgtcaaacctgtgtattcttaattggggtttcccccattaaagttacacaaaccaagggtgaaaccaagggaaccataaggtgctcctgtcacctgtcagaatgtctttacactttagttaaaaccctgtgtggcttacatgattctatcccctttaattaataaacttactttaaatctgcaatcacttcttcgtccttgtttttgtcaccatccagcagcaccaccttcccctcttcccaactttcatagcagattttccgaaaagaggcgtactttcgccacactttgaaaggaacgtttacttcactttcaagctctcccacaattttcattgcagatatgaaacgcgccaaagtggaagaaggatttaaccccgtttatccctatggatattccactccaactgacgtggctcctccctttgtagcctctgatggtcttcaagaaaacccacccggggtgttgtccttaaaaatagccaaacctctaactttcaacacctccaaagccctaagcttggcaattggcccgggtttaaaaattcaggatggtaaactagtgggggagggacaagcaattcttgcaaatttgccgctgcagatcaccaacaacacaatttcacttcgctttggaaacacacttgccctaaatgacaataatgaactgcaaacaacactccaatcttcatcacccctgaaaattaccgatcagactctgtcgctcaacataggtaacagccttgtagtaaaagatgacaaactagaaaacgcgcttaaagcatccctcccactttccattgctaacaacgccatcagcctcaacgtgggcacaggactaaccataaatggaaacactttacaagctgtgcccttaaatgctctaagtcccctaagtatttccaataataacatcagcctccgctacggcagctcactaacagtgctgaacaacgaactccaaagcaacctcacatttcacccacctttgaaactcaactctaacaactcagtttccctaaacactctatctccatttagaattgaaaatggctttttaacgctctatttgggcaccaaatctggcttgctagttgaaaacagcagcttaaaagttcagacgggctatggtctgcaagtaacagacaccaatgctctaaccttaagatacctccccccattaaccattcctgactcaggatcagaacaaggtattcttaaaataaacactggacagggcttaagtgtaagccaagctggaacgcttgaaacatccctaggaggtggattaaaacatgctgagggcaaaataaccattgatactggaaacggattaacactcagtgaaaacaaacttgccgtagcggcagggagtggcttaacattccgcgacggtgccttggtagctacagggtcatcatttacacaaaccctatggactacagcagatccatctccaaactgcaccgttctcgaggaacgtgatgctaaatttactttggctcttactattagcggaagtcaagtgctagggacagtgtctatggtaggagtaaaaggcgccctttttaataacataccatcctcttccatccaagtgcagctagactttgactctaccggagccctcttgagctcctcttcgctgaaaggatactggggatatcggcagggggtctcaatagacccttatcccataattaatgccttaaactttatgccaagttctctggcctatcccccagggcaaggtaatcaagcaaaatctaacgtgtatattcccacatatttacgaggaaacctccaaagaccaatagttttaaacatcacttttaataaccaaaacacggggttttccattagattttcatgggcaaatttaacaacaggagaaggatttgcaacaccagcatgcactttttcctacattgctgaacaacaataaactatgtaacccgaactgttattccgcctccacccttccatttcactttgtaaaccacccgctccaccttttcagcagtaaacaattgcaagtcagtaggggctacaaaacttttgggagttaaaatccacacgggttcttcacaggccaagcgagtatcagttacacttataaaaccatcactaacatcagacaaagacaaacatgagtccaaagcttccggttcaagttctgattttggttcactggcggcggggataacttcttccggagggttctccatctccttcatcagctcctccatgtccaccgtggtcatctgggacgagaacgacagttgtcatacacctcataagtcacaggtcgctgacgaacgtacaggtctcgaagaatgtcctgacgccgccgctcggcagcgctgggctgaaggcgaaatcgcccatgtctaacaatggccaacaccgcccgtctcatgaggcgccgagtcctcctagcgcaacagcgcatgcgcatttcgctgagactggcacaggagacacagcacaggaccaccagattgttcataatcccataagcgtgctgacaccagcccatgctaacaaattgcttcactattctagcatgtacatcatatctgatgttcaagtaaatcaaatgacgtcccctcatgtaaacacttcccacgtacaacacctccttgggcatctgataattaaccacctcccgataccaaagacatctctgattaatggccgccccgtacaccacccgattgaaccaagtggccaaaagaatcccccctgccatacactgcaaagagccggggcgtttacaatgacagtgcaaagtccacacctcgttgccatgaataacggaggaccgccttaagtcaatagtggcacagctaatacaaacatccaaatattgcttcatcaagtgccactcatatgaggtaagtatcatatcccagggaacgggccactccataaacactgcaaaacctacacatcccaccatcccccgcacggcactcacatcgtgcatggtgtgcatgtcacagtccggaagctgagaacaaggaaaagtctcaggagcattttcagagggcggtagtgggtactcctggtaggggttcagccggcaccggtatctcctcaccctctgggccatagcatacaagttgggacctaatctccaggtactttctgtatgaaaaccaggtgttttcccagcagtgtctgcgatgtcttctgtccccgcgacggtagcgctccttgcagtacgcgcggaacagccactcacgcaggcccaaaagacaattctcagcggaggttgacaagttaattcccctcagtctcaaggccaacagagtttcttccacagtagcataggccaaccccaaccaggaaacacaagctgacacgtcccgttcaacgggaggacaaggaagcagaggcagaggcataggcaaaagcaataaattttattccaactgatcacgtagcacttcaaacaccaggtcacgtagatggcatcgatcgtgggtttcctgatgaaacataacagcaaggtcaaaaatcaaacgattctcaagatgattaaccaccgcaggaattaggtcttcgacgcgtatatttaaaaacaccagcaatacaaaagcccggttttctcctggatctatcatagcagcacagtcatcaattagtcccaaataattttcccgcttccaatcagatataatttgcagaataattccctgtaaatccaacccggccatggcaaaaagctcggtcagggcgctctccacgtgcatgcgcaaacacaccctcatcttgtcaatccaaaaagtcttcttcctgagaaacctgcagcaaattaagaaccgccaggttaggctcgatgcctgcatcccggagcttcattctcaacatgcactgcaaatgctccagcaaatcagagcaacaattagcagccagctcatcgccagattccagttccggcgttcccacggcaactatcactcgaaacgtggggcaaattgaaacaacatgagctcccacgtgagcaaaagccgtggggcccgtgcaataatcgcagaaccaacggaaaaaagattgcagctcatgtttcaaaaagctctgcagatcaaaattcagctcatgcaaataacacagtaaggtttgcggtacagtaacagaaaaccacacgggtcgacgttcaaacatcttggcttacctaagaaaaaagaagcacattttaaaccacagttgcttcctgaacaggaggaaatatggtgcggcgtaaaaccaaacgtgccaccggatcgccggcggagccttggtaatacaaccagctgtggttaaacagcaatacttttaactcagcaacggttgaaggctccacataatcagcacccacaaaaatcccatcacgcacttgctcgcgcagggagctgaagtgagcagtatagccccagggcacccgaacgctaatctgcaagtacagcagagccaccccattaggtggaattacaaaatcagttggcgaaaacaacgtatacaccccggactgcaaaagctgctcaggcagacgcccccctggcccctcacggtacaccagcaaagcctcgggtaaagccgccatgccaagcgcttaccgtgccaagagcgactcagacggaaaagtgtggctgagacgctcagagcagcggctatatactctacctgtgacgtcaataaccgaaagtcaaaagttcacccggcgcgccccgaaaaacccgcgaaaatccacccaaaaagcccgcgaaaacacttccgcataaaatttccgggttcccgacgcgtcaccgccgcgcgacacgcccgccccgccccgcgctcctccccgaaacccgccgcgcccacttccgcgttcccgagccaaaggtcgcgtaactccgcccacctcatttgcatgttaactcggtcgccatcttgcggtgttatattgatgatg

SEQ ID NO: 36 - DNA, 885 bp, unidentified; identical to SEQ ID NO: 29
  source 1..885: mol_type = genomic DNA

(19) United States
(12) Patent Application Publication
(10) Pub. No.: US 2025/0066742 A1
(43) Pub. Date: Feb. 27, 2025

(54) VIRAL VECTOR AND RECOMBINANT SIMIAN ADENOVIRUS

(71) Applicant: Jinan University, Guangzhou (CN)

(72) Inventors: Qiwei ZHANG, Guangzhou (CN); Yiqiang LI, Guangzhou (CN); Wendong LAN, Guangzhou (CN); Junxian OU, Guangzhou (CN)

(73) Assignee: Jinan University, Guangzhou (CN)

(21) Appl. No.: 18/807,709

(22) Filed: Aug. 16, 2024

(30) Foreign Application Priority Data
Aug. 21, 2023 (CN) 2023110541953

(51) Int. Cl. C12N 7/00

(52) U.S. Cl. CPC C12N 7/00; C12N 2710/10321; C12N 2710/10341

(57) ABSTRACT

Provided are viral vector and recombinant Simian adenovirus. The viral vector carries at least one recombinant genome preparing from the Simian adenovirus genome by replacing E4orf6 gene, knocking out E3 gene, inserting EGFP gene at the loci of E3, or knocking out E1B55K gene. Recombinant Simian adenovirus could be preparing by transfecting the viral vector into host cells for packaging.

### CROSS-REFERENCE TO RELATED APPLICATIONS

This disclosure claims priority to Chinese Patent disclosure CN202311054195.3, filed with China Intellectual Property Office on Aug. 21, 2023, the entire contents of which are incorporated herein by reference.

### SEQUENCE LISTING

The sequence listing xml file submitted herewith, named “Sequence_Listing.xml”, created on Sep. 14, 2024, and having a file size of 81,125 bytes, is incorporated by reference herein.

### TECHNICAL FIELD

This disclosure relates to adenovirus, in particular to viral vector and recombinant Simian adenovirus.

### BACKGROUND

The statements herein provide background information relevant to the present disclosure only and do not necessarily constitute prior art.

Adenoviruses have a wide range of hosts in nature. ICTV (https://ictv.global/taxonomy) has classified adenovirus into 6 generas, and 87 species based on different hosts with adenoviruses, that include mammals, birds, poultry, fishes, reptiles, and amphibians. The mammalian adenoviruses are classified into the genus Mastadenovirus, of which Human adenovirus sp. (HAdVs) are divided into 7 species (HAdV A-G). Non-human primate-derived adenoviruses are usually called by a joint name Simian adenovirus sp. (SAdVs), that are divided into 9 total species (SAdV A-I), and at least 50 types have been identified so far.

Adenoviruses can infect many types of host, and generally does not integrate with the genome of the host. Adenoviruses can express exogenous genes with high efficiency. Adenoviruses include stable genome, and it can exist stably in liquid state and solid particle state. Adenoviruses are used as common viral vectors, and widely used for preparation of viral vaccines and gene therapy.

However, the wild genome of Adenovirus could only accommodate a exogenous gene that do not exceed 5% of the length of its genome. In addition, the positive rate of neutralizing antibody against HAdVs in serum is usually high in various races of crowd. This leads to higher pre-existing immunity of HAdV and lower efficiency of expressing exogenous genes, that also interferes with its use as a viral vector.

### SUMMARY

Embodiments disclose a viral vector. The viral vector carries at least one recombinant genome. The recombinant genome is prepared from a wild genome of Simian adenovirus by replacing the first E4orf6 gene of the wild genome with a second E4orf6 gene from a genome of Human adenovirus type 5. The wild genome has the nucleotide sequence set forth in SEQ ID NO: 35. The first E4orf6 gene has the nucleotide sequence 31414 to 32268 of SEQ ID NO: 35. The genome of Human adenovirus type 5 has the nucleotide sequence of AC_000008.1(NCBI RefSeq number). The second E4orf6 gene has the nucleotide sequence 33193 to 34077 of AC_000008.1 (set forth in SEQ ID NO: 36).

Embodiments disclose a recombinant Simian adenovirus possessing a recombinant genome. The recombinant genome is prepared from a wild genome of Simian adenovirus by replacing the first E4orf6 gene of the wild genome with a second E4orf6 gene from a genome of Human adenovirus type 5. The wild genome has the nucleotide sequence of SEQ ID NO: 35. The first E4orf6 gene has the nucleotide sequence 31414 to 32268 of SEQ ID NO: 35. The genome of Human adenovirus type 5 has the nucleotide sequence of AC_000008.1(NCBI RefSeq number). The second E4orf6 gene has the nucleotide sequence 33193 to 34077 of AC_000008.1 (set forth in SEQ ID NO: 36).

Embodiments disclose a viral vector. The viral vector carries at least one recombinant genome. The recombinant genome is prepared from a wild genome of Simian adenovirus by replacing a first E4orf6 gene of the wild genome with a second E4orf6 gene from a genome of Human adenovirus type 5, knocking out a E3 gene of the wild genome and inserting a EGFP gene at the original loci of the E3 gene. The wild genome has the nucleotide sequence set forth in SEQ ID NO: 35. The first E4orf6 gene has the nucleotide sequence 31414 to 32268 of SEQ ID NO: 35. The E3 gene has the nucleotide sequence 26084 to 29316 of SEQ ID NO: 35. The genome of Human adenovirus type 5 has the nucleotide sequence of AC_000008.1(NCBI RefSeq number). The second E4orf6 gene comprises the nucleotide sequence 33193 to 34077 of AC_000008.1 (set forth in SEQ ID NO: 36).

Embodiments disclose a recombinant Simian adenovirus possessing a recombinant genome. The recombinant genome is prepared from a wild genome of Simian adenovirus by replacing a first E4orf6 gene of the wild genome with a second E4orf6 gene from a genome of Human adenovirus type 5, knocking out a E3 gene of the wild genome of Simian adenovirus and inserting a EGFP gene at the original loci of the E3 gene. The wild genome has the nucleotide sequence set forth in SEQ ID NO: 35. The first E4orf6 gene has the nucleotide sequence 31414 to 32268 of SEQ ID NO: 35. The E3 gene has the nucleotide sequence 26084 to 29316 of SEQ ID NO: 35. The genome of Human adenovirus type 5 has the nucleotide sequence of AC_000008.1. The second E4orf6 gene comprises the nucleotide sequence 33193 to 34077 of AC_000008.1 (set forth in SEQ ID NO: 36).

Embodiments disclose a viral vector. The viral vector carries at least one recombinant genome. The recombinant genome is prepared from a wild genome of Simian adenovirus by replacing a first E4orf6 gene of the wild genome with a second E4orf6 gene from a genome of Human adenovirus type 5, knocking out a E3 gene of the wild genome, inserting a EGFP gene at the original loci of the E3 gene, knocking out a E1B55K gene of the wild genome. The wild genome has the nucleotide sequence set forth in SEQ ID NO: 35. The first E4orf6 gene has the nucleotide sequence 31414 to 32268 of SEQ ID NO: 35. The E3 gene has the nucleotide sequence 26084 to 29316 of SEQ ID NO: 35. The E1B55K gene has the nucleotide sequence 1821 to 3347 of SEQ ID NO: 35. The genome of Human adenovirus type 5 has the nucleotide sequence of AC_000008.1. The second E4orf6 gene has the nucleotide sequence 33193 to 34077 of AC_000008.1 (set forth in SEQ ID NO: 36).

Embodiments disclose a recombinant Simian adenovirus possessing a recombinant genome. The recombinant genome is prepared from a wild genome of Simian adenovirus by replacing a first E4orf6 gene of the wild genome with a second E4orf6 gene from a genome of Human adenovirus type 5, knocking out a E3 gene of the wild genome, inserting a EGFP gene at the original loci of the E3 gene, knocking out a E1B55K gene of the wild genome. The genome of Simian adenovirus has the nucleotide sequence set forth in SEQ ID NO: 35. The first E4orf6 gene has the nucleotide sequence 31414 to 32268 of SEQ ID NO: 35. The E3 gene has the nucleotide sequence 26084 to 29316 of SEQ ID NO: 35. The E1B55K gene has the nucleotide sequence 1821 to 3347 of SEQ ID NO: 35. The genome of Human adenovirus type 5 has the nucleotide sequence of AC_000008.1. The second E4orf6 gene has the nucleotide sequence 33193 to 34077 of AC_000008.1 (set forth in SEQ ID NO: 36).

### BRIEF DESCRIPTION OF DRAWINGS

FIG. 1 depicts a schematic diagram for construction of the first vector (pBRSAdVGZ3-12) according to embodiments.

FIG. 2 depicts a schematic diagram for construction of the third vector (pSAdV-Ad5E4orf6) from the first vector (pBRSAdVGZ3-12) and the second vector (pUC-Ad5E4orf6) according to embodiments.

FIG. 3 depicts a schematic diagram for construction of the fifth vector (pSAdV-ΔE3-Ad5E4orf6-EGFP) from the third vector (pSAdV-Ad5E4orf6) and the fourth vector (pUC-ΔE3-EGFP) according to embodiments.

FIG. 4 depicts a schematic diagram for construction of the seventh vector (pSAdV-ΔE3-ΔE1B55K-Ad5E4orf6-EGFP) from the fifth vector (pSAdV-ΔE3-Ad5E4orf6-EGFP) and the sixth vector(pUC-ΔE1B55K) according to embodiments.

FIG. 5 depicts a schematic diagram for construction of the eighth vector (pSAdV-ΔE3-EGFP) from the first vector (pBRSAdVGZ3-12) and the fourth vector (pUC-ΔE3-EGFP) according to embodiments.

FIG. 6 shows a microscopic image of Ad293 cells infected with the first virus according to embodiments.

FIG. 7 shows a white light (left) and a fluorescent light (right) of Ad293 cells infected with the fifth virus according to embodiments.

FIG. 8 shows white light (left) and fluorescent light (right) of Ad293 cells infected with the seventh virus according to embodiments.

FIG. 9a shows a curve of DNA content at various time points during the growth and propagation of various viruses according to embodiments. Herein, “SAdV GZ3-12 IN A549” refers to the growth of the first virus (the wild Simian adenovirus SAdV GZ3-12) in A549 cells. “SAdV GZ3-12 IN Ad293” refers to the growth of the first virus in Ad293 cells. “SAdV-Ad5E4orf6 IN A549” refers to the growth of the third virus (SAdV-Ad5E4orf6) in A549cells. “SAdV-Ad5E4orf6 IN Ad293” refers to the growth of the third virus in Ad293 cells. “SAdV-ΔE3-Ad5E4orf6-EGFP IN A549” refers to the growth of the fifth virus (SAdV-ΔE3-Ad5E4orf6-EGFP) in A549 cells. “SAdV-ΔE3-Ad5E4orf6-EGFP IN Ad293” refers to the growth of the fifth virus in Ad293 cells. “SAdV-ΔE3-ΔE1B55K-Ad5E4orf6-EGFP IN A549” refers to the growth of the seventh virus (SAdV-ΔE3-ΔE1B55K-Ad5E4orf6-EGFP) in A549 cells. “SAdV-ΔE3-ΔE1B55K-Ad5E4orf6-EGFP IN Ad293” refers to the growth of the seventh virus in Ad293 cells.

FIG. 9b shows a curve of the titer of live virus at various time points during the growth and propagation of various viruses according to embodiments. Herein, “SAdV GZ3-12 IN A549” refers to the growth of the first virus (the wild Simian adenovirus SAdV GZ3-12) in A549 cells. “SAdV GZ3-12 IN Ad293” refers to the growth of the first virus in Ad293 cells. “SAdV-Ad5E4orf6 IN A549” refers to the growth of the third virus (SAdV-Ad5E4orf6) in A549cells. “SAdV-Ad5E4orf6 IN Ad293” refers to the growth of the third virus in Ad293 cells. “SAdV-ΔE3-Ad5E4orf6-EGFP IN A549” refers to the growth of the fifth virus (SAdV-ΔE3-Ad5E4orf6-EGFP) in A549 cells. “SAdV-ΔE3-Ad5E4orf6-EGFP IN Ad293” refers to the growth of the fifth virus in Ad293 cells. “SAdV-ΔE3-ΔE1B55K-Ad5E4orf6-EGFP IN A549” refers to the growth of the seventh virus (SAdV-ΔE3-ΔE1B55K-Ad5E4orf6-EGFP) in A549 cells. “SAdV-ΔE3-ΔE1B55K-Ad5E4orf6-EGFP IN Ad293” refers to the growth of the seventh virus in Ad293 cells.

FIG. 10 shows a white light (A) and a fluorescent light (B) of SAdV-ΔE3-EGFP infected Ad293-E3 cells according to an embodiment. The left arrow indicates the aggregation of cell clusters expressed with green fluorescence under the field of view, and the right arrow indicates CPE phenomena of cells under white light.

FIG. 11a shows the FFU live virus titer growth curve of the eighth virus (SAdV-ΔE3-EGFP) in Ad293 cells and Ad293-E3 cells provided by embodiments.

FIG. 11b shows the DNA content variation curve of the eighth virus (SAdV-ΔE3-EGFP) in Ad293 cells and Ad293-E3 cells provided by embodiments.

FIG. 12a shows the DNA content variation curve of SAdV-ΔE3-EGFP and SAdV-ΔE3-Ad5E4orf6-EGFP respectively according to embodiments.

FIG. 12b shows the FFU live virus titer growth curve of SAdV-ΔE3-EGFP and SAdV-ΔE3-Ad5E4orf6-EGFP respectively according to embodiments.

### DETAILED DESCRIPTION OF THE EMBODIMENTS

The present disclosure will be described in further detail with reference to the following embodiments. It should be understood that the specific embodiments described herein are for purposes of illustration only and are not intended to limit the scope of the disclosure. The reagents not specifically and individually described in the present disclosure are all conventional reagents and are commercially available. Methods which are not specifically described in detail are all routine experimental methods and are known from the prior art.

It should be noted that, the terms “first”, “second”, and the like in the description and the claims of the present disclosure and the above drawings are used for distinguishing similar objects, and are not necessarily used for describing a particular sequence or order, and do not limit the technical features that follow. It is to be understood that the data so used may be interchanged where appropriate such that the embodiments of the disclosure described herein may be implemented in sequences other than those illustrated or otherwise described herein.

In China, the positive rate of neutralizing antibody in the serum against Human adenovirus type 5 (HAdV-5) is as high as 50%-80%, and in some areas of Africa, the positive rate is as high as 90% or even 100%. About 1184 serum samples from Guangdong province and Shandong province are reported that the positive rate of neutralizing antibody in the serum against Human adenovirus type 26 (HAdV-26) is as high as 47%, and the titer of the neutralizing antibody is between 200 and 1000, whereas the positive rate of neutralizing antibody against Human adenovirus type 35 (HAdV-35) is 15%, the titer is relatively low (72-200). Another study reports that the positive rate of neutralizing antibody in human serum against Human adenovirus type 3 (HAdV-3) is as high as 63%. A study in Korea include found that the positive rate of antibody against Human adenovirus type 55 (HAdV-55) are 18.8% in the general population, and 56.0% in the military personnel, respectively.

In contrast, there is a lower positive rate of neutralizing antibody against Simian adenovirus in healthy people. In a study in Thailand, the positive rate of neutralizing antibody in serum (n=113) against the Simian adenovirus RBR-7-10 is 6.8%, whereas in normal human serum (n=125), the positive rate is 0. Furthermore, in one study in Brazil, detection of 200 human serum include showed a positive rate of neutralizing antibody against Simian adenovirus AdC6 and Simian adenovirus AdC68 of 21% and 23%, respectively. In China, a similar epidemiological survey shows that the positive rate of neutralizing antibody against Simian adenovirus type 23 (SAdV-23) is between 6% and 20% in the serum samples from Guangzhou, Yichang, Xishan and Chengdu. In another study in Chongqing, the positive rate of neutralizing antibody against Simian adenovirus type 6 and Simian adenovirus type 7 in the serum of healthy volunteers were found to be 12.22% and 13.13%. Therefore, the positive rate of neutralizing antibody against Simian adenovirus is far lower than that of neutralizing antibody of HAdV in the population, and the pre-existing immunity rate of Simian adenovirus in the population is lower.

Based on the above, the inventor recombine the wild genome of Simian adenovirus, and prepare a recombinant Simian adenovirus. This recombinant Simian adenovirus avoid pre-existing immunity interference, and efficiently express exogenous gene. And this recombinant Simian adenovirus could be used as a safe replication-defective viral vector. And, this recombinant Simian adenovirus may be used as a viral vector for substituting the HAdV. Furthermore, this recombinant Simian adenovirus may be used as a vaccine vector and a therapeutic vector.

Embodiments disclose a viral vector. The viral vector carries at least one recombinant genome. The recombinant genome is prepared from a wild genome of Simian adenovirus by one or more options of:
replacing a first E4orf6 gene of the wild genome with a second E4orf6 gene from a genome of Human adenovirus type 5,
knocking out a E3 gene of the wild genome,
inserting a EGFP gene at the loci of the E3 gene, or
knocking out a E1B55K gene of the wild genome.

Embodiments provide a wild Simian adenovirus with a wild genome showing as SEQ ID NO: 35. This wild Simian adenovirus is screened and isolated from a monkey fecal sample. This wild Simian adenovirus is deposited in China Center for type Culture Collection (CCTCC) on Aug. 8, 2023, and named Simian Adenovirus SAdV GZ3-12 with the preservation number of CCTCC NO: V202385.

The wild genome has the nucleotide sequence of SEQ ID NO: 35. The first E4orf6 gene has the nucleotide sequence 31414 to 32268 of SEQ ID NO: 35. The E3 gene has the nucleotide sequence 26084 to 29316 of SEQ ID NO: 35. The E1B55K gene has the nucleotide sequence 1821 to 3347 of SEQ ID NO: 35. A E1 gene is located at the nucleotide sequence 491 to 3347 of SEQ ID NO:35. A E4 gene is located at the nucleotide sequence 31100 to 33741 of SEQ ID NO:35.

The genome of Human adenovirus type 5 (HAdV-5) has the nucleotide sequence of AC_000008.1 (NCBI RefSeq number). The second E4orf6 gene has the nucleotide sequence 33193 to 34077 of AC_000008.1(set forth in SEQ ID NO:36). The Enhanced Green Fluorescent Protein is coded by the EGFP gene.

In some embodiments, the recombinant genome prepared from the wild genome of by replacing the first E4orf6 gene of the wild genome with the second E4orf6 gene from the genome of Human adenovirus type 5.

In some embodiments, the recombinant genome prepared from the wild genome by replacing the first E4orf6 gene of the wild genome with the second E4orf6 gene from the genome of Human adenovirus type 5, knocking out a E3 gene of the wild genome, and inserting a EGFP gene at the original loci of the E3 gene.

In some embodiments, the recombinant genome prepared from the wild genome by replacing the first E4orf6 gene of the wild genome with the second E4orf6 gene from the genome of Human adenovirus type 5, knocking out the E3 gene of the wild genome, inserting the EGFP gene at the original loci of the E3 gene, and knocking out the E1B55K gene of the wild genome.

In some embodiments, the recombinant genome prepared from the genome of Simian adenovirus by knocking out the E3 gene of the wild genome, inserting the EGFP gene at the original loci of the E3 gene.

In some embodiments, the viral vector further has the region of a basal vector.

In some embodiments, the basal vector is selected from a group consisting of pBR322, pUC18, pUC19, pBluescript or pcDNA3.1.

Embodiments provide a recombinant Simian adenovirus possessing the recombinant genome providing with above embodiments.

Embodiments provide a method of preparing the viral vector providing with above embodiments. The method include: preparing a first vector, preparing a second vector and simultaneously transforming the first vector and the second vector into a Escherichia coli to obtain a third vector. The first vector carries at least one wild genome of Simian adenovirus. The second vector carries the E4orf6 gene from HAdV-5 genome. The Escherichia coli expresses a recombinase. So that the third vector carries the recombinant genome possessing the second E4orf6 at the original loci of the first E4orf6 gene.

In some embodiments, the method further include: preparing a fourth vector, and simultaneously transforming the third vector and the fourth vector into the Escherichia coli to obtain a fifth vector. The fifth vector is configured to knock out the E3 gene of the wild genome of Simian adenovirus and insert the EGFP gene. So that the fifth vector carries the recombinant genome possessing the second E4orf6 at the original loci of the first E4orf6 gene, and the EGFP gene at the original loci of the E3 gene.

In some embodiments, the fourth vector carries a nucleotide sequence consisting of an upstream region of the E3 gene from the wild genome, a downstream region of the E3 gene from the wild genome, the EGFP gene, a CMV promoter and a CMV enhancer.

In some embodiments, the method further include: preparing a sixth vector, and simultaneously transforming the fifth vector and the sixth vector into the Escherichia coli to obtain a seventh vector. The sixth vector is configured to knock out the E1B55K gene of the wild genome of Simian adenovirus. So that the seventh vector carries the recombinant genome possessing the second E4orf6 at the original loci of the first E4orf6 gene, the EGFP gene at the original loci of the E3 gene, and with deleting original loci of E1B55K gene.

In some embodiments, the sixth vector carries a nucleotide sequence consisting of an upstream region of E1B55K gene from the wild genome, and a downstream region of E1B55K gene from the wild genome.

In some embodiments, the method further include: simultaneously transforming the first vector and the fourth vector into the Escherichia coli to obtain a eighth vector. So that the eighth vector carries the recombinant genome possessing the EGFP gene at the original loci of the E3 gene.

The replication-defective recombinant Simian adenovirus could be prepared by transfecting the viral vectors said above into cells for packaging.

In some embodiments, a first virus (named SAdV GZ3-12) could be prepared by transfecting the first vector into host cells for packaging.

In some embodiments, a third virus (named SAdV-Ad5E4orf6) could be prepared by transfecting the third vector into host cells for packaging.

In some embodiments, a fifth virus (named SAdV-ΔE3-Ad5E4orf6-EGFP) could be prepared by transfecting the fifth vector into host cells for packaging.

In some embodiments, a seventh virus (named SAdV-ΔE3-ΔE1B55K-Ad5E4orf6-EGFP) could be prepared by transfecting the seventh vector into host cells for packaging.

In some embodiments, a eighth virus (named SAdV-ΔE3-EGFP) could be prepared by transfecting the eighth vector into host cells for packaging.

In some embodiments, the host cells are selected from the group of HEK293, Ad293, Ad293-E3 or A549.

Embodiments provide uses of the viral vectors disclosed above. These uses includes preparation of vaccines against Simian adenovirus, vaccines against Human adenovirus type 5, drugs for genic curing diseases caused by Simian adenovirus or Human adenovirus type 5.

This disclosure will be described in further detail with reference to the accompanying drawings, in which the disclosure is not limited to the embodiments shown.

The proteins, nucleic acids, vectors, Escherichia coli, Simian adenovirus, Human adenovirus type 5 and host cells of the disclosure may be natural, chemical synthetic, or produced from prokaryotic or eukaryotic hosts (e.g., bacteria, yeast, plants) by using recombinant techniques and genetic engineering means.

Embodiments provide primers (shown in Table 1) for amplifying those genes and some functional regions according to the wild genome (SEQ ID NO: 35). Embodiments also provide primers (shown in Table 1) for amplifying the second E4orf6 gene from the HAdV-5's genome (AC_000008.1). These primers have been synthesized by the Sonchaceae Biotechnology Co., ltd., and dissolved at a working concentration of 10 μM with ddH₂O, and stored at 4° C. or −20° C.

TABLE 1
Primers list

| Name | Sequence (5′→3′) |
|---|---|
| PBR-SWAI- | gttaactcggtcgccatcttgcggtgttatattgatgatgatttaaatctcatgtttgacagcttatcatcg, |
| ITR-F | SEQ ID NO: 1, the underlined sequences is homologous to pBR322 (basal |
| | vector) |
| PBR-SWAI- | gttaactcggtcgccatcttgcggtgttatattgatgatgatttaaatcttgaagacgaaagggcctcg, SEQ |
| ITR-R | ID NO: 2, the underlined sequences is homologous to pBR322 |
| Ad5E4orf6-F | ctcctccatgtccaccgtggctacatgggggtagagtcat, SEQ ID NO: 3, the underlined is |
| | homologous sequence |
| Ad5E4orf6-R | taggtcccaacttgtatgctatgactacgtccggcgttcca, SEQ ID NO: 4, the underlined is |
| | homologous sequence |
| EGFP-F | agttattaatagtaatcaattac, SEQ ID NO: 5 |
| EGFP-R | tgcagtgaaaaaaatgc, SEQ ID NO: 6 |
| E1B55K-UP-F | gattacgccaagcttctaagcttccgggtgactca, SEQ ID NO: 7, the underlined |
| | sequences is homologous to pUC19 |
| E1B55K-UP-R | catggtacctgaaaactactcctccgctggagggt, SEQ ID NO: 8, the underlined |
| | sequence is an upstream region of E1B55K that is homologous to pUC19 |
| E1B55K- | gagtagttttcaggtaccatgagcggatcaagcagcc, SEQ ID NO: 9, the underlined |
| DOWN-F | sequence is an upstream region of E1B55K is homologous to the downstream |
| | region of E1B55K |
| E1B55K- | acggccagtgaattcgatatcttgcgcaacctgctttccac, SEQ ID NO: 10, the underlined |
| DOWN-R | sequence is a downstream region of E1B55K that is homologous to pUC19 |
| E3L-F | aaaacgacggccagtgaattcgatatcgattcccacgccttac, SEQ ID NO: 11, the |
| | underlined sequence is homologous to pUC19 |
| E3L-R | attgattactattaataacttcgccgtagtaccaagt, SEQ ID NO: 12, the underlined |
| | sequence is is homologous to EGFP |
| E3R-F | gcatttttttcactgctctgcaatcacttcttegtcc, SEQ ID NO: 13, the underlined |
| | sequence is homologous to EGFP |
| E3R-R | taaaacgacggccagtgGATATCccatagcccgtctgaac, SEQ ID NO: 14, the |
| | underlined sequence is a downstream region of E3 that is homologous to |
| | pUC19 |
| E4L-F | accatgattacgccaGAATTCatctgtctgcagctactttcatc, SEQ ID NO: 15, the |
| | underlined sequence is homologous to pUC19 |
| E4L-R | atgactctacccccatgtagccacggtggacatggaggag, SEQ ID NO: 16, the underlined |
| | sequence is a homologous sequence of E4L that is homologous to the |
| | upstream region of HAdV-5 E4orf6 |
| E4R-F | ggaacgccggacgtagtcatcatagcatacaagttgggac, SEQ ID NO: 17, the underlined |
| | sequence is a homologous sequence of E4R that is homologous to the |
| | downstream region of HAdV-5 E4orf6 |
| E4R-R | aaatcagagcaacaattagctctagGAATTCcactggccgtcgttttacaa, SEQ ID NO: 18, |
| | the underlined sequence is homologous to a region of HAdV-5 E4orf6 |
| Hexon-F | gccccartgggcrtacatgcacatc, SEQ ID NO: 19 |
| Hexon-R | agcacsccscgratgtcaaag, SEQ ID NO: 20 |
| Elcom-F | agatggcgaccgagttaa, SEQ ID NO: 21 |
| Elcom-R | agatgtgctgatagtagcgtag, SEQ ID NO: 22 |
| pUC19-F | ttcacccacctttgaaactcGAATTCactggccgtcgttt, SEQ ID NO: 23, the underlined |
| | is homologous sequence, the uppercase is the EcoRI site |
| pUC19-R | tagcgttagttttgccatttTCTAGAgtcgacctgcaggca, SEQ ID NO: 24, |
| | the underlined is homologous sequence, the uppercase is the Xbal site |
| plenti-E3F | agacaccgactctagatatcatgactgatgtcgagcccgc, SEQ ID NO: 25, the underlined |
| | is the sequence that E3 is ovelapped to plenti-Flag |
| plenti-E3R | ttgtagtcagcccgggatccttatagatgaaagtagctgc, SEQ ID NO: 26, the underlined is |
| | the sequence that E3 is ovelapped to plenti-Flag |

### Prepare a First Vector and a First Virus

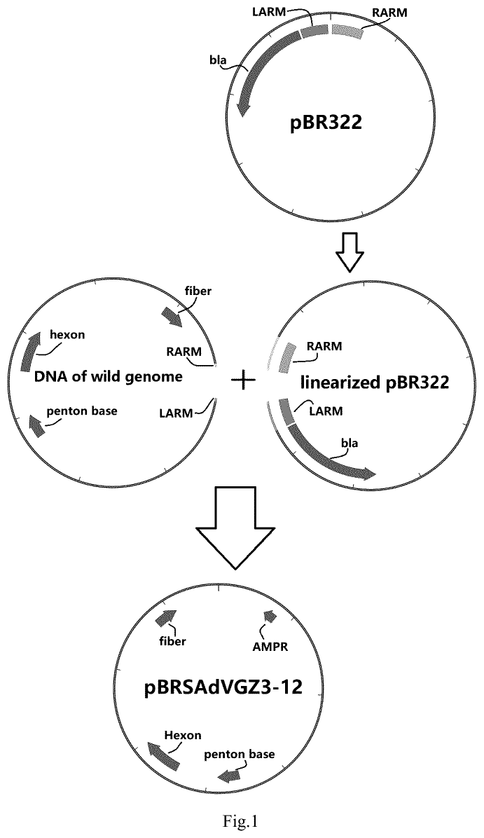

As shown in FIG. 1, the first vector (named pBRSAdVGZ3-12) was prepared by Gibson Assembly. The first virus (named SAdV GZ3-12) was prepared by transfecting and assembling the first vector in host cells. The process included the following steps:

Step (1): A 5×reaction buffer was prepared as shown in Table 2, and packaged and stored at −20° C. for use.

TABLE 2
5× reaction buffer for Gibson
Assembly at constant temperature

| Reagent | Dosage | |
|---|---|---|
| 1 M Tris-HCl, pH = 7.5 | 1 | mL |
| 2 M MgCl₂ | 50 | μL |
| 10 mM dNTP | 200 | μL |
| 1 M DTT | 100 | μL |
| PEG-8000 | 0.5 | g |
| 100 mM NAD | 100 | μL |

| | |
|---|---|
| ddH₂O | add to 2 mL |

Step (2): An enzymatic mixture for Gibson Assembly was prepared as shown in Table 3, packaged and stored at −20° C. for use.

TABLE 3
Enzymatic mixture for Gibson Assembly

| Reagent | Dosage | |
|---|---|---|
| 5× reaction buffer | 40 | μL |
| 10 U/μL T5 exonuclease | 0.1 | μL |
| 40 U/μL Taq DNA ligase | 20 | μL |
| 2 U/μL phusion DNA polymerase | 2.5 | μL |
| ddH₂O | 87.4 | μL |

Step (3): A reaction system for Gibson assembly was prepared according to Table 4. Herein, the linearized pBR322 was amplified from pBR322 (PM11779, PERFEMIKER) by using PBR-SWAI-ITR-F/PBR-SWAI-ITR-R as a primer pair in a PCR.

TABLE 4
Reaction system for Gibson Assembly

| Reagent | Dosage |
|---|---|
| DNA of wild genome | 4 equivalents |
| linearized pBR322 | 1 equivalent |
| enzymatic mixture | add to 20 μL |

In the step (3), the reaction system for Gibson Assembly was placed in a refrigerator at 4° C. for 3 hours, and then at 50° C. for 1 hour. Finally, the mixture was taken out and stored at −20° C.

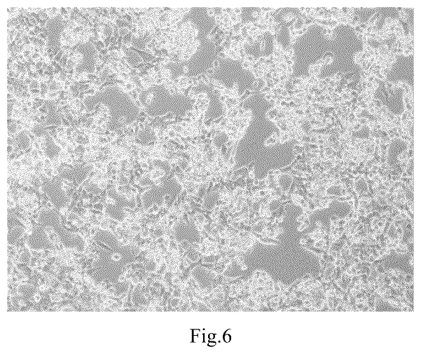

As shown in FIG. 6, the first vector had transfected into Ad293 cells, and there was a CPE effect, and the first virus was rescued.

It is proved that the first vector include the whole wild genome of SAdV GZ3-12, and is infectious, and is able to assemble live virus in cells.

### Prepare a Third Vector and a Third Virus

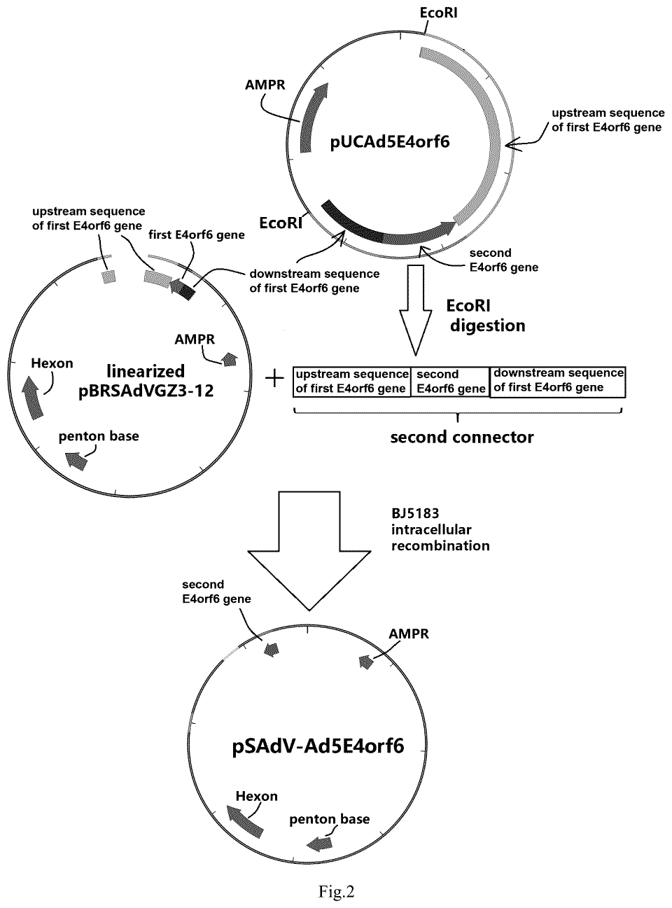

As shown in FIG. 2, the third virus was prepared by: preparing the first vector with carrying one wild genome; preparing a second vector with carrying a second connector that was used to substitute the first E4orf6 gene with a second E4orf6 gene; and simultaneously transforming the first vector and the second vector into Escherichia coli to obtain the third vector. Herein, the Escherichia coli could express a recombinase. And the third virus was prepared by transfecting the third vector into HEK293 and packaging virus.

It is proved that the third virus is a defective Simian adenovirus in replication. Both the third vector and the third virus carry a recombinant genome including the second E4orf6 gene locating at original loci of the first E4orf6 gene from the wild genome of SEQ ID NO: 35.

In an embodiment, the second connector was consisted of an upstream sequence of first E4orf6 gene, the second E4orf6 gene, and a downstream sequence of first E4orf6 gene.

In a process, the second vector was prepared by:
(1) amplifying a pUC19 plasmid (SKU number: B610005-0050, manufactured and bioengineered (Shanghai) Co., ltd.) by using pUC19-F and pUC19-R as a primer pair in a PCR;
(2) digesting the amplified pUC19 plasmid with EcoRI to obtain a linearized fragment of pUC19 plasmid;
(3) amplifying the upstream homologous sequence (SEQ ID NO: 27) of first E4orf6 gene from the nucleotide sequence 28912 to 31413 of SEQ ID NO: 35 with primers including E4L-F and E4L-R;
(4) amplifying the downstream homologous sequence (SEQ ID NO: 28) of first E4orf6 gene from the nucleotide sequence 32269 to 33068 of SEQ ID NO: 35 with primers including E4R-F and E4R-R;
(5) amplifying the second E4orf6 gene (SEQ ID NO: 29) from the HAdV-5's genome with primers including Ad5E4orf6-F and Ad5E4orf6-R;
(6) simultaneously adding the linearized fragment of pUC19 plasmid, the upstream homologous sequence of first E4orf6 gene, the downstream homologous sequence of first E4orf6 gene and the second E4orf6 gene into a connection system for seamless clone, and reacting for 30 min at 37° C.;
(7) after transforming the product of step (6) into competent E. coli DH5a, screening positive clones from agar plates by ampicillin, verifying and sequencing these positive clones to get the second vector (named pUC-Ad5E4orf6).

In a process, the third vector was prepared by:
(1) linearizing the first vector with SpeI enzyme to expose its homology arm, digesting the second vector with Pad enzyme and EcoRI enzyme to recovering the second connector;
(2) homologous recombining the linearized first vector and the second connector, and transforming the recombinant into competent E. coli BJ5183;
(3) screening positive clones from agar plates by ampicillin, and verifying by PCRs with primers of Hexon-F/Hexon-R and primers of Ad5E4orf6-F/Ad5E4orf6-R to obtain double positive clones, and double positive plasmids could be exacted from the double positive clones;
(4) digesting the double positive plasmids with four restriction enzymes, and testing the enzymaitc digestion by agarose gel electrophoresis. If the bands were consistent with the SnapGene mimic, the plasmid was judged to be correct, and the correct plasmid was verified to be the third vector (named pSAdV-Ad5E4orf6).

In a process, the third virus was prepared by:
(1) amplifying the third vector, and digesting the amplified products into the wild genome and a backbone of pBR322 with SwaI enzyme;
(2) transfecting the wild genome into Ad293 cells through liposomes for packaging;
(3) harvesting viruses from the transfected cell culture with generating typical CPE phenomena such as rounding, fusion, grape string shape and the like, repeatedly freezing, thawing and harvesting the supernatant from the thawing, and transfecting the supernatant into Ad293 cells again for enlarge culturing the viruses; and verifying the third virus (named SAdV-Ad5E4orf6) by enzyme digestion, PCR or other methods.

### Prepare the Fifth Vector and the Fifth Virus

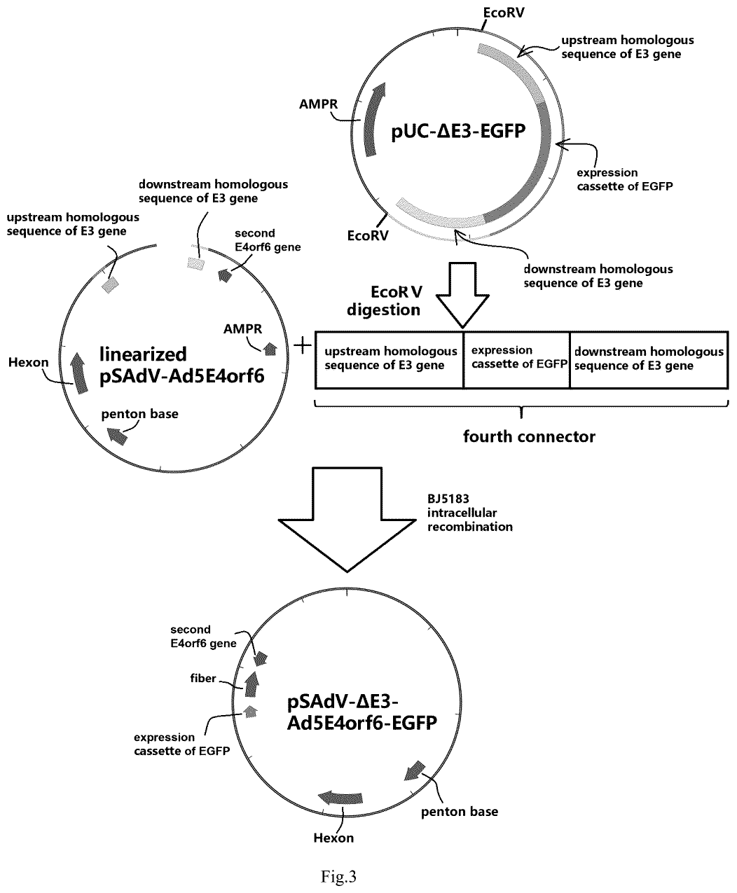

As shown in FIG. 3, the fifth virus was prepared by: providing the third vector and the fourth vector with carrying a fourth connector for replacing the E3 gene of the wild genome with a EGFP gene; simultaneously transforming the third vector and the fourth vector into E. coli BJ5183 to obtain the fifth vector; transfecting the fifth vector into cells HEK293 and packaging virus to obtain the fifth virus.

It is proved that the fifth virus is a defective Simian adenovirus in replication. Both the fifth vector and the fifth virus possess a recombinant genome including the second E4orf6 gene locating at the original loci of the first E4orf6 gene of the wild genome of SEQ ID NO: 35, and the EGFP gene locating at the original loci of the E3 gene of the wild genome of SEQ ID NO: 35.

In an embodiment, the fourth connector (SEQ ID NO: 30) was consisted of an upstream homologous sequence of E3 gene, an expression cassette of EGFP (consisting of a CMV promoter, a CMV enhancer and a EGFP gene), a downstream homologous sequence of E3 gene.

In an embodiment, the construction process of the fourth vector specifically included:
(1) amplifying the upstream homologous sequence of E3 gene from the nucleotide sequence 25756 to 26416 of SEQ ID NO: 35 with primers including E3L-F and the E3L-R;
(2) amplifying the downstream homologous sequence of E3 gene from the nucleotide sequence 29332 to 30352 of SEQ ID NO: 35 with primers including E3R-F and the E3R-R;
(3) amplifying a pEGFP-C1 plasmid (SKU number: VT1118, order@youbio.cn) with primers including EGFP-F and EGFP-R to obtain the expression cassette of EGFP, this expression cassette of EGFP could be used as a indicator for virus packaging and observation; meanwhile, restriction site was added in EGFP-F and EGFP-R to convenient substitute EGFP;
(4) adding the linearized fragment of pUC19, the upstream homologous sequence of E3 gene, the downstream homologous sequence of E3 gene, and the expression cassette of EGFP into a seamless cloning reaction system at the same time, reacting for 30 min at 37° C., and screening and verifying to obtain the fourth vector (named pUC-ΔE3-EGFP).

In an embodiment, the construction process of the fifth vector specifically included:
(1) linearizing the third vector with Pad to expose its homology arm, digesting the fourth vector with EcoRV to recover the fourth connector;
(2) simultaneously transforming the linearized third vector and the fourth connector into competent E. coli BJ5183, homologous recombining and coating on agar plates, screening positive clones by PCRs with primers of Hexon-F/Hexon-R and EGFP-F/EGFP-R, and extracting the fifth vector plasmid (pSAdV-ΔE3-Ad5E4orf6-EGFP) from these positive clones.

In an embodiment, the recombinant genome from Simian adenovirus was recovered by digesting the fifth vector with SwaI. And the recombinant genome from Simian adenovirus was transfected into Ad293 cells by liposomes to package viruses. The state of cell growth was observed daily while the intensity of fluorescence produced by the cells was observed by fluorescence microscope.

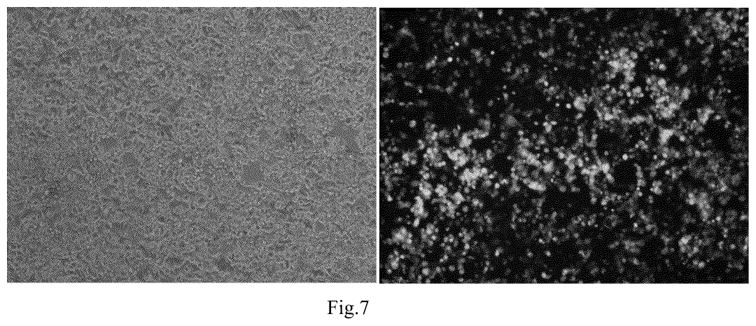

As shown in FIG. 7, if aggregated cells appear in the fluorescent field, the fifth virus is harvested and the expansion culture is continued. The fifth virus (named SAdV-ΔE3-Ad5E4orf6-EGFP) is verified by PCR, enzymatic digestion or other methods.

### Prepare the Seventh Vector and the Seventh Virus

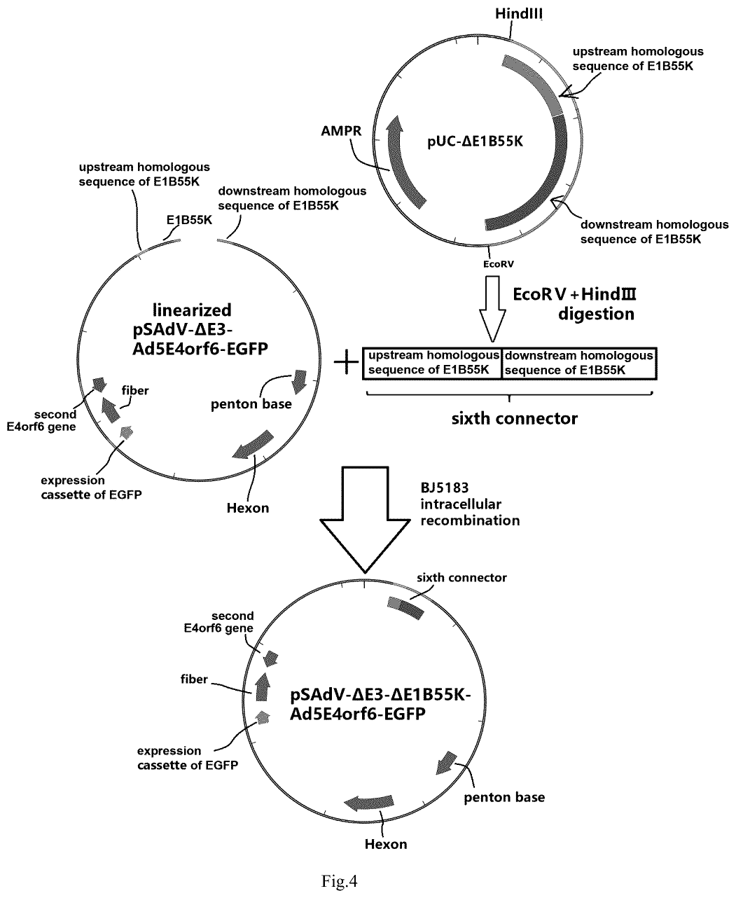

In an embodiment, as shown in FIG. 4, the seventh vector was prepared by: providing the fifth vector, and a sixth vector with carrying a sixth connector for knocking out the E1B55K gene of Simian adenovirus SAdV GZ3-12; simultaneously transforming the fifth vector and the sixth vector into E. coli BJ5183 that could express a recombinase. And the seventh virus could be prepared by transfecting the seventh vector into cells HEK293 and packaging viruses.

It is proved that the seventh virus is a defective Simian adenovirus in replication. Both the seventh vector and the seventh virus possess a recombinant genome including the second E4orf6 gene locating at the original loci of the first E4orf6 of the wild genome of SEQ ID NO: 35, a EGFP gene locating at the original loci of the E3 gene of the wild genome of SEQ ID NO: 35.

In an embodiment, the sixth connector was consisted of an upstream homologous sequence of E1B55K gene and a downstream homologous sequence of E1B55K gene.

In an embodiment, the sixth vector was prepared by:
(1) amplifying the upstream homologous sequence (SEQ ID NO: 31) of E1B55K gene from the nucleotide sequence 1358 to 2072 of SEQ ID NO: 35 with primers including E1B55K-UP-F and E1B55K-UP-R;
(2) amplifying the downstream homologous sequence (SEQ ID NO: 32) of E1B55K gene from the nucleotide sequence 3422 to 4509 of SEQ ID NO: 35 with primers including E1B55K-DOWN-F and E1B55K-DOWN-R, herein these primers had restriction sites for inserting a target sequence into the loci of E1B55K gene; and the 3′ end of the upstream homologous sequence of E1B55K gene had a 15 nt homologous sequence with the 5′ end of the downstream homologous sequence of E1B55K gene;
(3) adding the linearized fragment of pUC19, the upstream homologous sequence of E1B55K gene and the downstream homologous sequence of E1B55K gene into a seamless cloning reaction system, reacting for 30 min at 37° C., screening and verifying to get the sixth vector (named pUC-ΔE1B55K).

In an embodiment, the construction process of the seventh vector specifically includes:
(1) digesting the fifth vector with PmeI to linearly expose its homology arm; and digesting the sixth vector with EcoRI and HindIII to recover the sixth connector;
(2) simultaneously transforming the digested fifth vector and the seventh connector into competent E. coli BJ5183 to homologous recombine; and coating the recombinant on agar plates; and screening positive clones by using primers Hexon-F/Hexon-R and E1com-F/E1com-R in PCRs; and extracting the seventh vector plasmid (named pSAdV-ΔE3-ΔE1B55K-Ad5E4orf6-EGFP) from these positive clones.

In an embodiment, a recombinant genome was recovered from the amplified seventh vector by a enzymatic digestion with SwaI. The recovered was transfected into Ad293 cells by liposomes, packaging to obtain the seventh virus. The state of cell growth was observed daily while the intensity of fluorescence produced by the cells was observed by fluorescence microscope.

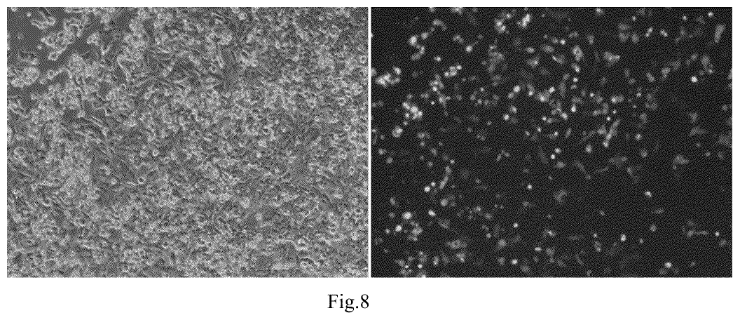

As shown in FIG. 8, if aggregated cells appear in the fluorescent field, the seventh virus (named SAdV-ΔE3-ΔE1B55K-Ad5E4orf6-EGFP) is harvested and the expansion culture is continued. The seventh virus could be verified by PCR, enzymatic digestion or other methods.

### Prepare the Eighth Vector and the Eighth Virus

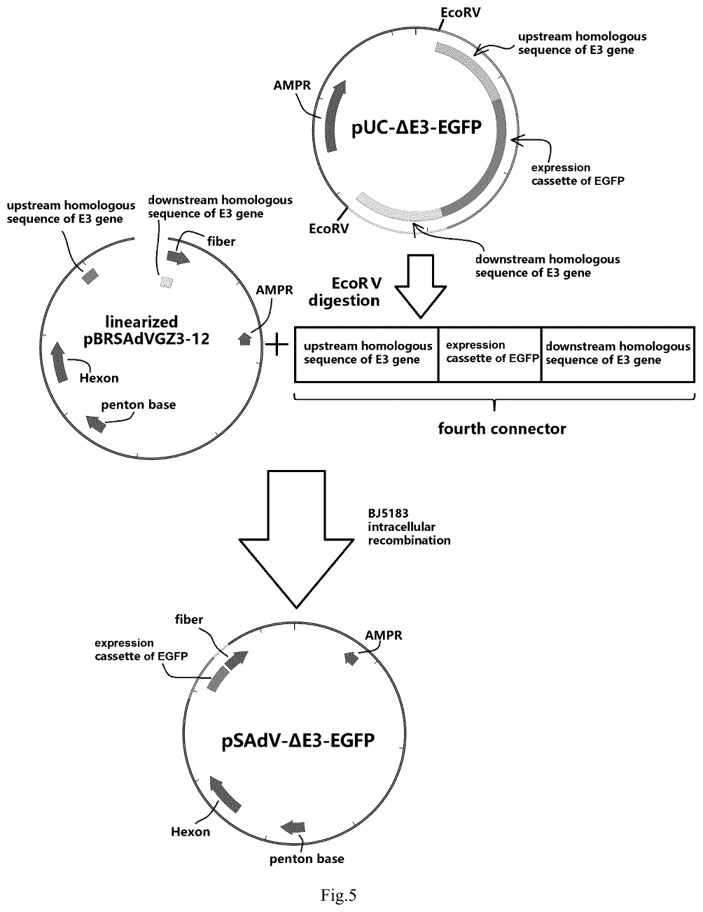

In an embodiment, as shown in FIG. 5, the eighth vector was prepared by: digesting the first vector and the fourth vector with enzymes respectively; and transforming into Escherichia coli BJ5183. And the eighth virus could be prepared by transfecting the eighth vector into cells HEK293 and packaging virus.

In an embodiment, the linearized first vector and the linearized fourth vector were transformed into competent Escherichia coli BJ5183, and coated on plates. Positive clones were screened by using primers Hexon-F/Hexon-R and EGFP-F/EGFP-R in Colony PCR. The eighth vector could be extracted from these positive clones.

In an embodiment, the recombinant genome was recovered from the amplified eighth vector by a enzymatic digestion with SwaI. The recombinant genome was transfected into Ad293 cells and Ad293-E3 cells by liposomes respectively, and the eighth virus could be packaged. The state of cell growth was observed daily while the intensity of fluorescence produced by the cells was observed by fluorescence microscope.

Herein, the Ad293-E3 cell could express the E3 gene of Simian adenovirus SAdV GZ3-12. In some embodiments, the Ad293-E3 cell may be prepared by:
(1) amplifying the E3 gene from the wild genome of Simian adenovirus SAdV GZ3-12 with primers including plenti-E3F and plenti-E3R, the amplified product could contain five sequences of CR1-α, CR1-β, RID-α, RID-0 and E3-14.7K; herein, an enzymatic cut site of EcoRI and a sequence of Kozak may be sequentially added at the 5′ end of plenti-E3F, and an enzymatic cut site of HindIII may be added at the 5′ end of plenti-E3R;
(2) digesting a plenti-Flag plasmid (Addgene Co.) and the amplified product of the E3 gene with HindIII and EcoRI; recovering a backbone of plenti-Flag and a fragment of E3 after digesting;
(3) ligating the backbone of plenti-Flag with the fragment of E3 at a molar ratio of 1:3 by T4 ligase; reacting for 12 hours at 16° C.; and transforming the ligated into competent E. coli DH5α, screening positive clones and exacting plasmids (named plenti-E3) from the positive clones;
(4) inoculating 5×10⁵ Ad293 cells into one well of a six well plate;
(5) simultaneously transfecting vectors of psPAX2 (Invitrogen), pMD2. G (Addgene), and plenti-E3 at a mass ratio of 4:3:1 into the Ad293 cells completely adherent to the well with a total transfected dosage of 8 μg per well; after transfection, cultivating the transfected cells in a cell incubator;
(6) harvesting the supernatant from the cultivated of 72 hours, filtering through a 0.45 μm filter, and stored at −80° C.;
(7) adding 1 mL of the supernatant from the step (6) into one well with containing completely adherent 5×10⁵ Ad293 cells, substituting the virus solution with complete medium after 2 hours;
(8) after 72 hours of cultivating as step (7), removing the medium, and adding a complete medium with containing 2 μg/mL puromycin to each well, and substituting these medium every two days;
(9) after 5 days of cultivating as step (8), passaging at a normal cell process, purifying and screening cells by using a 96-well plate to obtain the E3 expressed cells of Ad293 (Ad293-E3).

In one test embodiment, the ability of Ad293-E3 to transcribe E3 was tested. The test process specifically include: extracting RNA of Ad293-E3 with Trizol reagent, reversely transcribing by a enzyme kit (TAKARA company, Reverse Transcriptase M-MLV (RNase H)—) with random primers. Herein, the reaction system of reverse transcription was incubated at 37° C. for 10 min and reacted at 42° C. for 1 hour. The reaction was terminated by treating at 70° C. for 15 min, and rapidly taken out and placed on ice for cooling for 15 min. And the obtained solution of cDNA was used for PCR as shown in Table 5.

TABLE 5

| Reagent | volume(μL) |
|---|---|
| CDNA | 6 |
| 5× M-ML V Buffer | 2 |
| dNTP Mixture(10 mM) | 0.5 |
| RNase Inhibitor(40 U/μL) | 0.25 |
| Reverse Transcriptase M-MLV | 0.25 |
| (RNase H-)(200 U/μL) | |
| RNase free H₂O | 1 |
| Total | 10 |

### Prepare Virus Samples in Large Quantities

In an embodiment, the cell fluids of above embodiments were harvested, and washed with PBS, and were subjected to the steps of −80° C. (freezing) and 37° C. steps (thawing) for three times, which caused the virus to be sufficiently released from the cells. Supernatants was collected by centrifuging the virus-releasing liquid at 12000×g for 2 min. And samples of the first virus, the third virus, the fifth virus, the seventh virus and the eighth virus could be collected from the supernatants.

Test 1: qPCR to Detect the Content of Viral DNA

Samples to be tested: the first virus sample, the third virus sample, the fifth virus sample and the seventh virus.

Detection method:

(1) Hexon-F and Hexon-R were dissolved and diluted to a working concentration of 10 μM.

(2) A standard pUC-hexon plasmid (shown in SEQ ID NO: 33) was removed from a refrigerator, thawed on ice, and diluted with sterilized and deionized water to working concentrations of 10²-10⁸ copies/μL for use as standard samples. Meanwhile, 2 μL of the sample to be tested was used as a template. The construction method of pUC-hexon included: amplifying 300 bp (shown as SEQ ID NO: 34) of HAdV-5 Hexon gene by using a primer pair Hexon-F/Hexon-R in a PCR; and connecting the amplified product to the pUC19 plasmid; and verifying to obtain pUC-hexon. The concentration of pUC-hexon was determined according to the formula: plasmid concentration (ng/μL)×10⁻⁹×6.02×10²³/Molecular weight of double-stranded DNA=copies of pUC-hexon (copies/μL). The copies could be converted and recorded by this formula. This standard plasmid may be diluted at concentrations of 10²-10⁸ copies/μL for use, and stored at −20° C.

(3) A reaction system of qPCR may contain 1 mL TB Green™ Premix Ex TaqMT II (Tli RNaseH Plus)(2×), 40 μL ROX Reference Dye II (50×), 80 μL 10 μM upstream primer HexF, 80 μL of 10 μM downstream primer HexR and 600 μL sterilized water in a sterilized 2 mL EP tube. The mixture of these system was briefly centrifuged, dispensed into special eight-well tubes each of which was added 18 μL. And 2 μL template DNA was added to each tube. This step was performed entirely on ice and the liquid from the step was collected by a short centrifugation to the bottom of the tube.

(4) Amplification and detection procedures were set up on the QuantStudio™ Real-Time PCR Software system: pre-denaturation at 95° C. for 5 min; 40 cycles of amplification (95° C. for 10 s, 55° C. for 30 s; 72° C. for 20 s); amplification for melting curve (95° C. for 15 s, 60° C. for 1 min, 95° C. for 15 s).

(5) A standard curve could be made by combining the amplified curve, the melting curve to adjust the background value, the copies and CT value of the standard DNA. The CT value of samples to be tested (the first virus sample, the third virus sample, the fifth virus sample and the seventh virus sample) could be calculated by the standard value. The copies of samples to be tested could be calculated by the CT value. The growth curve of the virus could be plotted with a ordinate (Log 10 Genomic DNA copies/mL) according to the copies of samples, and an abscissa according to different time points of virus infection.

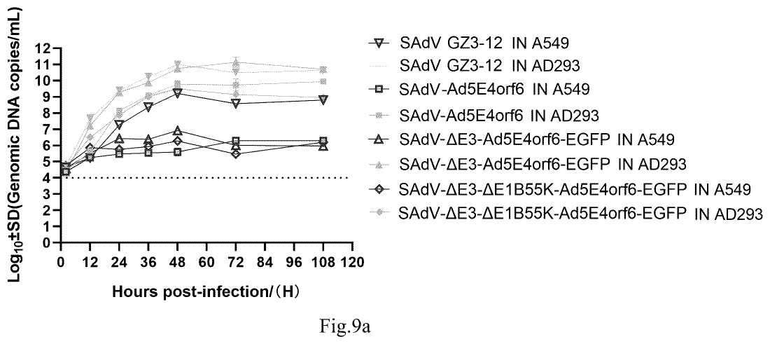

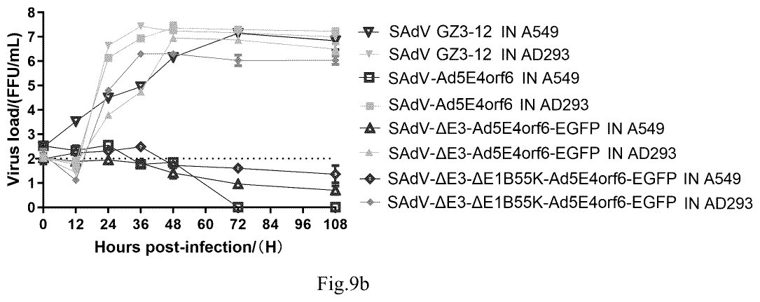

As shown in FIG. 9A, the third, fifth and seventh virus have lower content of viral DNA in A549, and higher content of viral DNA in Ad293. Whereas, the first virus (wild-type) possesses a higher content of viral DNA in both A549 and Ad293. As shown in FIG. 9A and FIG. 9B, the third, the fifth and the seventh virus normally replicate in the Ad293, but only infect A549 without replicating (show the same trend in other cells Caco2, Hela, Huh7, HepG2, Vero). It indicates that the third, the fifth and the seventh virus are defective in replication, and could only replicate in the specific cell Ad293 cell. And that also suggests the third, the fifth and the seventh vector are safety vectors.

### Test 2: Direct Immunofluorescence for Detecting Live Virus Number

Sample to be tested: the first virus sample, the third virus sample, the fifth virus sample, and the seventh virus sample

The test process included:
(1) diluting the first, third, fifth and seventh virus samples with DMEM medium at a 10-fold dilution, and dripping to a cell suspension with 10⁵ cells/mL of Ad293 after removing medium; and setting four auxiliary wells for each dilution, and negative wells and positive wells for control simultaneously;
(2) placing all wells in a cell incubator at 37° C. with 5% CO₂ for 2 hours, and then adding 100 μL of maintenance medium to all wells and continuously cultivating at 37° C. with 5% CO₂ for 40 hours; and then removing the medium from the cultivated cell solution, drying for 10 min in a biosafety cabinet;
(3) adding 100 μL pre-chilled anhydrous methanol at 4° C. to each well; and then placing the well plate at −20° C. for 10 min to fix the cells; removing methanol, adding 100 μL PBST each well, shaking slowly on a shaker for 10 min, and washing the plate 3 times repeatedly;
(4) adding 100 μL PBS with containing 1% BSA to each well, and slowly shaking at 37° C. for 30 min; washing the plate 3 times again with PBST, discarding the wash solution; and adding 50 μL FITC-labeled adenovirus universal antibody (Ruida Biotechnology Co., guangzhou) with dilution of 1:500 to each well, wrapping the well plate completely with aluminum foil paper for protecting from light, and slowly shaking on a shaking table for 1 hour.
(5) after removing the antibody, washing the plate 3 times with PBST, removing the wash solution, and back-buckling the well plate to a clean absorbent paper to suck the wash solution.
(6) And observing the fluorescence of cell by fluorescence microscope. If the cells in the negative wells did not have green fluorescence and most of the cells in the positive wells distribute fluorescence, the sample wells to be tested could be continuously observed and the number of cells with fluorescence could be counted. The live virus titer of Samples to be tested could be calculated by a formula: Fluorescence Formation Unit (FFU)/mL=10×sample dilution×average positive cell numbers of GFP.

As shown in FIG. 9B, the third, fifth and seventh virus had lower viral titers in A549 and higher viral titers in Ad293. Whereas the first virus (wild-type) had higher viral titers in both A549 and Ad293.

### Test 3: Replication Kinetics Test of the Eighth Virus

Test samples could be prepared by:
(1) inoculating the eighth virus into 5×10⁴ Ad293 cells and Ad293-E3 cells on a 12-well plate respectively, and three wells for each cell.
(2) after the cells grown into monolayer cells, infecting SAdV-ΔE3-EGFP with MOI=0.1 to Ad293 and Ad293-E3, respectively; cultivating at 37° C. for 2 hours, with gentle shaking every half hour.
(3) after 2 hours of virus infection of cells, removing the virus solution in the well plate; and after washing once with PBS buffer, adding 1 mL of maintenance medium to each well; and continuously placing the mixture at 37° C. and 5% CO₂ for cultivating.
(4) collecting the Ad293 liquid and the Ad293-E3 liquid respectively, washing by PBS, and placing in the steps of −80° C. (freezing) and 37° C. (thawing) for 3 times, and centrifuging at 12000×g for 2 min, and taking the supernatant respectively, thus obtaining the eighth virus sample.

Immunofluorescence test: the test method was the same as that above test 2.

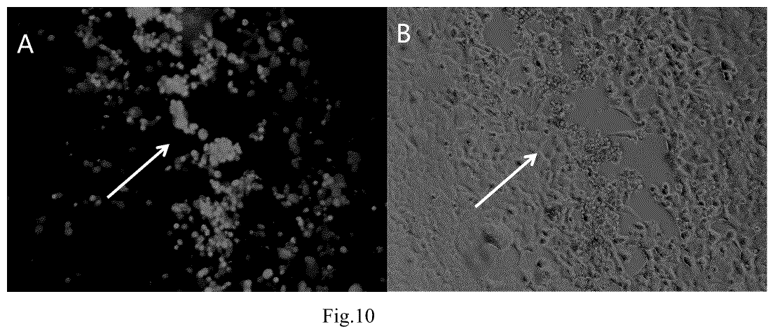

As shown in FIG. 10, the eighth virus could be able to infect Ad293 and Ad293-E3.

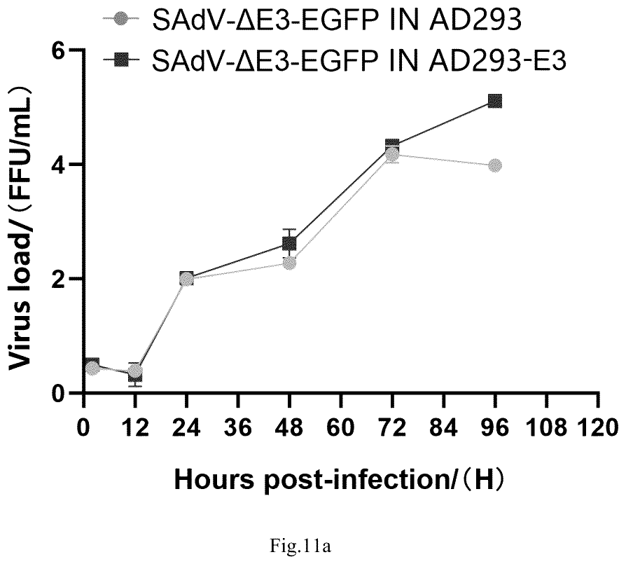

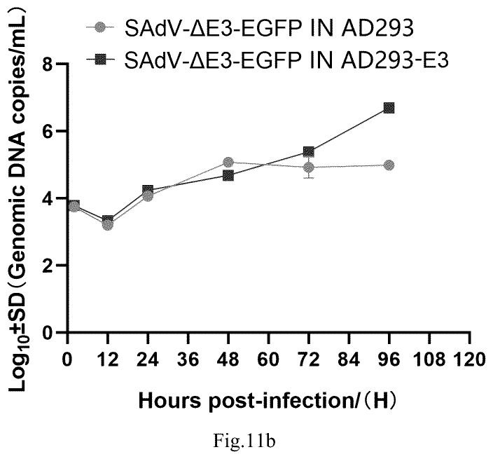

As shown in FIG. 11, the eighth virus could be able to infect and replicate in both Ad293 and Ad293-E3.

### Test 4: Test of Replication Difference Between the Eighth Virus and Fifth Virus

The replication difference between the eighth virus and fifth virus could be tested by:
(1) inoculating 10⁴ Ad293-E3 in each wells of a 12-well plate, and cultivating in a cell incubator for 12 hours;
(2) after grown into monolayer cells, infecting SAdV-ΔE3-EGFP with MOI 0.1 to the monolayer cells in three wells, and infecting SAdV-ΔE3-Ad5E4orf6-EGFP with MOI 0.1 to the monolayer cells in other three wells; cultivating at 37° C. for 2 hours with gentle shaking every half hour;
(3) Examining the viral DNA content and the live virus titer of the fifth virus and the eighth virus by the methods provided from test 1 and test 2, respectively.

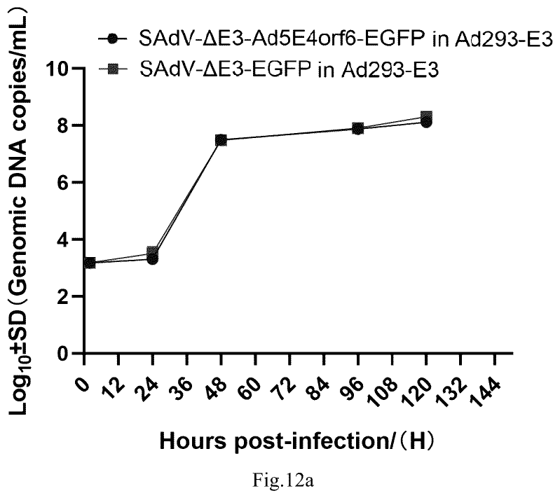

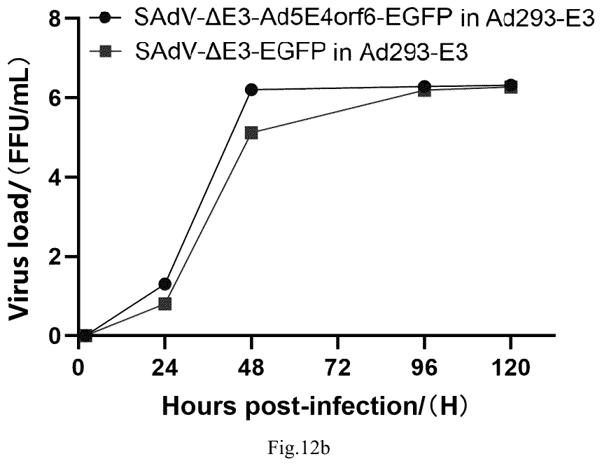

As shown in FIG. 12, the eighth virus is not significantly different from the fifth virus in replication ability.

The above is only the preferred embodiments of this disclosure and is not intended to limit this disclosure. Any modification, equivalent replacement, improvement, etc., made within the spirit and principle of this disclosure shall be included in the scope of this disclosure.

What is claimed is:

1. A viral vector comprising at least one recombinant genome, said recombinant genome is prepared from a wild genome of Simian adenovirus by replacing the first E4orf6 gene of said wild genome with a second E4orf6 gene;
said wild genome of Simian adenovirus comprises the nucleotide sequence set forth in SEQ ID NO: 35, said first E4orf6 gene comprises the nucleotide sequence 31414 to 32268 of SEQ ID NO: 35;
said second E4orf6 gene comprises the nucleotide sequence set forth in SEQ ID NO: 36.

2. A viral vector comprising at least one recombinant genome, said recombinant genome is prepared from a wild genome of Simian adenovirus by replacing a first E4orf6 gene of said wild genome with a second E4orf6 gene, knocking out a E3 gene of said wild genome and inserting a EGFP gene at the original loci of said E3 gene;
said wild genome comprises the nucleotide sequence set forth in SEQ ID NO: 35, said first E4orf6 gene comprises the nucleotide sequence 31414 to 32268 of SEQ ID NO: 35, said E3 gene comprises the nucleotide sequence 26084 to 29316 of SEQ ID NO: 35;
said second E4orf6 gene comprises the nucleotide sequence set forth in SEQ ID NO: 36.

3. A viral vector comprising at least one recombinant genome, said recombinant genome is prepared from a wild genome of Simian adenovirus by replacing a first E4orf6 gene of said wild genome with a second E4orf6 gene, knocking out a E3 gene of said wild genome, inserting a EGFP gene at the original loci of said E3 gene, knocking out a E1B55K gene of said wild genome;
said wild genome comprises the nucleotide sequence set forth in SEQ ID NO: 35, said first E4orf6 gene comprises the nucleotide sequence 31414 to 32268 of SEQ ID NO: 35, said E3 gene comprises the nucleotide sequence 26084 to 29316 of SEQ ID NO: 35, said E1B55K gene comprises the nucleotide sequence 1821 to 3347 of SEQ ID NO: 35;
said second E4orf6 gene comprises the nucleotide sequence set forth in SEQ ID NO: 36.
